(19) United States
(12) Patent Application Publication
(10) Pub. No.: US 2021/0038494 A1
(43) Pub. Date: Feb. 11, 2021

(54) FOAMING CLEANSER COMPOSITIONS CONTAINING A NON-POLAR OIL AND AMPHIPHILIC POLYMER

(71) Applicant: Lubrizol Advanced Materials, Inc., Cleveland, OH (US)

(72) Inventors: Xianghua Qu, Shanghai (CN); Kwan Hyun Cho, Gyeonggi-do (KR); Brian D. Figura, Cleveland, OH (US); Yan Zhang, Shanghai (CN)

(21) Appl. No.: 16/981,349

(22) PCT Filed: Mar. 11, 2019
(86) PCT No.: PCT/US2019/021536

§ 371 Date: Sep. 16, 2020

Related U.S. Application Data
(60) Provisional application No. 62/643,959, filed on Mar. 16, 2018.

(51) Int. Cl. A61K 8/81; A61K 8/36; A61K 8/06; A61Q 5/12; A61K 8/49; A61K 8/31; A61K 8/89; A61Q 5/02; A61K 8/44

(52) U.S. Cl. CPC A61K 8/8152; A61K 8/361; A61K 8/062; A61Q 5/12; A61K 2800/596; A61K 8/31; A61K 8/89; A61Q 5/02; A61K 8/442; A61K 8/4913

(57) ABSTRACT

The disclosed technology relates to a phase stable personal care cleansing composition containing high amounts of a non-polar oil. The compositions are high foaming and provide conditioning benefits to the scalp and skin. The composition comprises:
a) water;
b) a crosslinked nonionic amphiphilic polymer;
c) a non-polar oil phase;
d) at least one fatty acid soap; and
e) an optional detersive surfactant other than d).

### TECHNOLOGICAL FIELD

The present technology relates to a personal care cleansing composition, and a method of using said composition for cleansing, conditioning, and moisturizing a keratinous substrate, such as the skin or hair. More particularly, the present technology is directed to a cleansing formulation composition comprising: at least one fatty acid, a non-ionic cross-linked rheology modifier, a non-polar oil phase, and water, wherein the formulation is stable. When the formulation is used to cleanse the skin, the formulation provides conditioning and moisturization of the keratinous substrate, while also providing enhanced foaming and lather.

### BACKGROUND

The development of liquid personal cleansers (including, without limitation, body washes, facial washes, shampoos, liquid hand cleansers, and intimate cleansers) is often driven by the challenge of meeting conflicting consumer demands. At present, an increasing demand from consumers is for cleansing formulations which can deliver increasing levels of moisturization. A common means of providing moisturization is to include an emulsified or stabilized oil in the cleansing formulation to reduce water loss from the skin and improve skin health. While it can be challenging to stabilize the oil required to deliver moisturization, it is increasing challenging when the demand for moisturization is coupled with a requirement that the product demonstrate desirable foam and lather. As oils are known anti-foaming agents, the dual requirements of delivering moisturization and desirable lather erect a significant challenge to formulators of personal care cleansing products.

Efforts have been made to reduce the use of body cleansers that contain harsh synthetic surfactants by substituting the surfactant with liquid soaps derived from fatty acid salts. Liquid fatty acid soap compositions are known in the art. These soaps have been widely employed for many years as effective mild general all-purpose body cleansers. Fatty acid soaps are formulated with a myriad of different ingredients to obtain the desired cleansing effect and the requisite physical property parameters so that they can be easily stored and dispensed in a convenient manner. Fatty acid soaps must have the appropriate rheology characteristics to be flowable when dispensed from the product container but of a sufficient viscosity not run from the skin when applied to the body. In addition, today's consumer is looking for additional benefits beyond the basic cleansing effects brought about a traditional soap product. Efforts are continually being made to make improvements in product function and aesthetics by incorporating various adjuncts into the formulation such as moisturizers, emollients, colorants, opacifiers, perfumes, antioxidants, antibacterial agents, and the like to name a few. It has also been popular to incorporate water insoluble moieties such as microcapsules, beads, and pearlescent agents into the soap composition for delivery of actives to the skin and for product aesthetics.

In order to achieve the desired rheology profiles and to disperse the multitude of different ingredients within the soap composition, synthetic rheology modifying polymers and synthetic surfactants have been employed in an attempt to obtain a composition which is stable with respect to viscosity and visual phase homogeneity over a period of time and a wide range of temperatures. These parameters are particularly significant for liquid compositions wherein the large quantity of water in the formulation makes the establishment of a stable composition more difficult, particularly when substantially water insoluble adjuncts are dispersed in the formulation.

U.S. Patent Application Pub. No. U.S. 2000/09116074 discloses a stable lathering cleansing formulation comprising (a) from about 5 to 30 parts lipid skin moisturizing agent; (b) a water dispersible gel forming polymer wherein said polymer is an anionic, nonionic, cationic or hydrophobically modified polymers, selected from the group consisting of cationic polysaccharides of the cationic guar gum class with molecular weights of 1,000 to 3,000,000; anionic, cationic or nonionic homopolymers derived from acrylic or methylacrylic acid; anionic, cationic or nonionic cellulose resins; cationic copolymers of dimethyldialkylammonium chloride or acrylic acid; cationic homopolymers of dimethyldialkylammonium chloride; cationic polyalkylene or ethoxypolyalkylene imines; polyethylene glycol of molecular weight from 100,00 to 4,000,000; and mixtures thereof; (c) from about 5 part to about 30 parts of a synthetic surfactant; (d) from about 0 part to about 15 part of a C₈ to C₁₄ fatty acid soap; (e) from about 0.5 parts to about 6 parts of an additional non-polymeric thickener; and (f) water. However, the disclosure does not contemplate the wider range of oil concentration that is enabled by the present technology. Furthermore, the disclosure does not contemplate the use of a nonionic, amphiphilic, crosslinked polymer to stabilize an oil phase and facilitate the moisturization of the skin.

U.S. Patent Application Pub. No. U.S. 2007/0213243 discloses a stable soap composition comprising: (a) a crosslinked acrylic copolymer (INCI name: Acrylates Copolymer); (b) a fatty acid soap; (c) an alkalizing agent; (d) an optional surfactant; (e) an optional humectant; (f) an optional emollient; and (g) water. The composition is stabilized with the acrylic copolymer and subsequently back-acid treated with the acidifying agent to obtain compositions that are storage and phase stable over a wide temperature range.

International Pub. No. WO 2015/038601 discloses a method for mitigating pruritus caused by prolonged exposure to low humidity conditions by bathing with a liquid soap composition thickened with the crosslinked acrylic copolymer (INCI name: Acrylates Copolymer) disclosed in U.S. 2007/0213243.

The Acrylates Copolymer disclosed in U.S. 2007/0213243 and WO 2015/038601 is prepared from (meth)acrylic acid, a C₁ to C₅ alkyl ester of (meth)acrylic acid and a polyunsaturated crosslinker. The disclosed thickener requires neutralization with an alkalizing agent and optional back-acidification with an acidifying agent to build viscosity. Accordingly, the disclosed thickening agents are pH dependent meaning that the thickening mechanism relies on changing the pH of the composition in which they are contained to build viscosity.

International Pub. No. WO 2014/099573 discloses conventionally crosslinked nonionic amphiphilic polymers and their use as ocular and/or dermal irritation mitigants in surfactant containing compositions. The polymers mitigate irritation of the skin and eyes caused by harsh synthetic detersive surfactants contained in personal care cleansing compositions. The disclosed amphiphilic polymers provide tailored yield stress properties (the ability to stably suspend insoluble materials) to cleansing formulations across a wide pH range. The disclosed polymers do not require neutralization with a base or an acid in order to activate the thickening mechanism. In other words, the thickening mechanism is independent of pH.

International Pub. No. WO 2015/095286 discloses a nonionic amphiphilic polymer rheology modifier crosslinked with amphiphilic crosslinking agent or a mixture of an amphiphilic crosslinking agent and a conventional crosslinking agent. The disclosed amphiphilic polymers provide tailored yield stress properties to surfactant containing cleansing formulations across a wide pH range.

While International Pub. Nos. WO 2014/099573 and WO 2015/095286 disclose fatty acid salt soaps among a myriad of anionic surfactants useful in the disclosed surfactant containing compositions, there is no recognition that a cleansing composition comprising a high concentration of oily materials can be stabilized with a combination of fatty acid soap(s) and a crosslinked nonionic amphiphilic polymer, while providing conditioning and moisturization to keratinous substrate, and also providing enhanced foaming and lather.

Surprisingly, we have found that personal liquid cleansers formulated with a fatty acid soap and a crosslinked nonionic amphiphilic polymer enables the stabilization of formulations containing elevated levels of oil, thus enabling increased moisturization benefits, while also producing consumer-pleasing foam and lather. In particular, it has been discovered that the soap based cleansing composition disclosed herein can be employed during normal bathing intervals as an effective cleanser and moisturizer for the scalp and skin to ameliorate dry scalp and skin caused by prolonged exposure to low humidity environments.

### SUMMARY OF THE TECHNOLOGY

In accordance with a general embodiment of the present technology, a liquid cleansing composition comprising a fatty acid salt soap base selected from at least one fatty acid salt, a crosslinked nonionic amphiphilic emulsion polymer, a non-polar oil phase, and water utilized during normal bathing intervals to cleanse the scalp or skin (keratinous substrates) provides increased skin moisturization and improved skin health while also providing consumer desirable foam and lather.

In accordance with another embodiment of the present technology, the cleansing formulation is shelf-stable and comprises a soap base selected from at least one fatty acid salt, a crosslinked nonionic amphiphilic emulsion polymer, water, an oil or lipid phase, and optionally a synthetic surfactant selected from an anionic surfactant (different than a fatty acid soap), an amphoteric surfactant, and mixtures thereof.

In accordance with another embodiment of the technology there is provided a cleansing composition for moisturizing the skin comprising:
a) a soap comprising at least one fatty acid salt;
b) a crosslinked nonionic amphiphilic emulsion polymer prepared from:
   i. from about 35% to about 55% by weight of at least one C₁ to C₅ hydroxyalkyl ester of (meth)acrylic acid;
   ii. from about 10% to about 50% by weight of at least one monomer selected from a C₁ to C₅ alkyl (meth)acrylate; and
   iii. from about 0.1% to about 20% by weight of at least one associative monomer and/or a semi-hydrophobic monomer (wherein all monomer weight percentages are based on the weight of the total monounsaturated monomers);
   vi. from about 0.01 to about 5 parts by weight of at least one polyunsaturated crosslinker monomer (based on 100 parts by wt. of the monounsaturated monomers utilized to prepare the polymer);
c) a non-polar oily phase;
d) water; and
e) optionally at least one surfactant (different than a fatty acid soap).

In accordance with another embodiment of the technology there is provided cleansing composition for moisturizing the skin comprising:

a) a soap comprising at least one fatty acid salt;

b) a crosslinked nonionic amphiphilic emulsion polymer prepared from
i. from about 40% to about 50%, or from about 42% to about 48%, or from about 44 to 46 by weight of 2-hydroxyethyl methacrylate;
ii. from about 10% to about 40%, or from about 12% to about 35%, or from about 15% to about 25% by weight of ethyl acrylate;
iii. from about 10% to about 35%, or from about 12% to about 30%, or from about 15% to about 25% by weight of butyl acrylate;
iv. from about 0.5% to about 18%, or about 1%, 2%, 3%, 4%, 5%, 6%, 7%, 8%, 9% or 10% to about 15% by weight of an associative monomer selected from behenyl ethoxylated methacrylate (based on 100 parts by wt. of the monounsaturated monomers utilized to prepare the polymer); and
v. from about 0.1, 0.2, 0.3, 0.4, 0.5, 0.6, 0.7, 0.8 or 0.9 to about 1, or about 1.5, 2 or 3 to about 5 parts by wt. of at least one polyunsaturated crosslinker monomer selected from a polyunsaturated amphiphilic crosslinking monomer (based on 100 parts by wt. of the monounsaturated monomers utilized to prepare the polymer);

c) a non-polar oily phase;

d) water; and

e) optionally at least one surfactant (different than a fatty acid soap).

In accordance with still another embodiment of the technology there is provided a cleansing composition for moisturizing the skin comprising:

a) a soap comprising at least one fatty acid salt;

b) a crosslinked nonionic amphiphilic emulsion polymer prepared from
i. about 44% by weight of 2-hydroxyethyl methacrylate;
ii. about 35% by weight of ethyl acrylate;
iii. about 15% by weight of butyl acrylate;
iv. about 6% by weight of behenyl ethoxylated methacrylate (based on 100 parts by wt. of the monounsaturated monomers utilized to prepare the polymer); and
v. from about 0.5 to about 2 parts by wt. of at least one polyunsaturated amphiphilic crosslinker monomer (based on 100 parts by wt. of the monounsaturated monomers utilized to prepare the polymer);

c) a non-polar oily phase;

d) water; and

e) optionally at least one surfactant (different than a fatty acid soap).

In accordance with still another embodiment of the technology there is provided a cleansing composition for moisturizing the skin comprising:

a) a soap comprising at least one fatty acid salt

b) a crosslinked nonionic amphiphilic emulsion polymer prepared from
i. about 45% by weight of 2-hydroxyethyl methacrylate;
ii. about 15% by weight of ethyl acrylate;
iii. about 25% by weight of butyl acrylate;
iv. about 15% by weight of behenyl ethoxylated methacrylate (based on 100 parts by wt. of the monounsaturated monomers utilized to prepare the polymer); and
v. from about 0.5 to about 2 part by wt. of at least one polyunsaturated amphiphilic crosslinker monomer (based on 100 parts by wt. of the monounsaturated monomers utilized to prepare the polymer);

c) a non-polar oily phase;

d) water; and

e) optionally at least one surfactant (different than a fatty acid soap).

In accordance with still another embodiment of the technology there is provided a cleansing composition for moisturizing the skin comprising:

a) a soap comprising at least one fatty acid salt;

b) a crosslinked nonionic amphiphilic emulsion polymer prepared from
i. about 45% by weight of 2-hydroxyethyl methacrylate;
ii. about 20.5% by weight of ethyl acrylate;
iii. about 27.5% by weight of butyl acrylate;
iv. about 7% by weight of behenyl ethoxylated methacrylate (based on 100 parts by wt. of the monounsaturated monomers utilized to prepare the polymer); and
v. from about 0.1 to about 1 part by wt. of at least one polyunsaturated amphiphilic crosslinker monomer (based on 100 parts by wt. of the monounsaturated monomers utilized to prepare the polymer);

c) a non-polar oily phase;

d) water; and

d) optionally at least one surfactant (different than a fatty acid soap).

In accordance with still another embodiment of the technology there is provided a method for cleansing and moisturizing the skin comprising applying to the scalp and/or skin any of the disclosed cleansing compositions enumerated above and rinsing the applied composition from the scalp and/or skin.

While it can be challenging to stabilize the oil required to deliver moisturization in a cleansing product, it is increasingly challenging when the demand for moisturization is coupled with a requirement that the product demonstrate desirable foam and lather. As oils are known anti-foaming agents, the dual requirements of delivering moisturization and desirable lather erect a significant challenge to formulators of personal care cleansing products.

Embodiments of the technology disclosed herein are based on the surprising discovery that a cleansing composition comprising a non-polar oily phase, at least one fatty acid salt soap, a crosslinked nonionic amphiphilic emulsion polymer, water, and optionally, at least one surfactant (different than the fatty acid soap) is stable over long periods of time, moisturized the scalp and/or skin, while providing desirable foam and lather.

### Description of Exemplary Embodiments

Aspects according to the present technology are described hereinafter. Various modifications, adaptations or variations of such exemplary aspects described herein may become apparent to those skilled in the art as such are disclosed. It will be understood that all such modifications, adaptations or variations that rely on the teachings of the present technology, and through which these teachings have been advanced in the art, are considered to be within the scope and spirit of the present technology.

As used herein, the prefix “(meth)acryl” includes “acryl” as well as “methacryl”. For example, the term “(meth)acrylic acid” includes both acrylic acid and methacrylic acid.

The term “nonionic” as used herein encompasses both a monomer, monomer composition or a polymer polymerized from a monomer composition devoid of ionic or ionizable moieties (“nonionizable”), and a “substantially nonionic” monomer, monomer composition or polymer polymerized from a monomer composition.

An ionizable moiety is any group that can be made ionic by neutralization with an acid or a base.

An ionic or an ionized moiety is any moiety that has been neutralized by an acid or a base.

By “substantially nonionic” is meant that the monomer, monomer composition or polymer polymerized from a monomer composition contains less than or equal to 15 wt. % in one aspect, less than or equal to 10 wt. % in another aspect, less than or equal to 5 wt. % in still another aspect, less than or equal to 3 wt. % in a further aspect, less than or equal to 1 wt. % in a still further aspect, less than or equal to 0.5 wt. % in an additional aspect, less than or equal to 0.1 wt. % in a still additional aspect, and less than or equal to 0.05 wt. % in a further aspect, of an ionizable and/or an ionized moiety. Those of ordinary skill in the art will recognize that depending on the commercial source, some nonionic monomers may contain residual amounts of a monomer with ionic or ionizable character. The amount of residual monomer in a nonionic monomer composition that contains ionic or ionizable moieties can range from 0, 0.05, 0.5, 1, 2, 3, 4, or 5 to 15 wt. % based on the weight of the nonionic monomer.

The phrase “at least one” means one or more of a particular component and thus includes individual components as well as mixtures/combinations of individually recited components.

The methods, polymers, components, and compositions of the present technology may suitably comprise, consist of, or consist essentially of the components, elements, steps, and process delineations described herein. The technology illustratively disclosed herein suitably may be practiced in the absence of any element, component or step which is not specifically disclosed herein.

Unless otherwise stated, all percentages, parts, and ratios expressed herein are based upon the total composition weight of the soap cleansing composition.

When referring to a specified monomer(s) that is incorporated into a polymer of the disclosed technology, it will be recognized that the monomer(s) will be incorporated into the polymer as a monomer residue(s) derived from the specified monomer(s) (e.g., a repeating unit).

Here, as well as elsewhere in the specification and claims, individual numerical values (including carbon atom numerical values), or limits, can be combined to form additional non-disclosed and/or non-stated ranges.

The headings provided herein serve to illustrate, but not to limit the disclosed technology in any way or manner.

The selection and the amounts of the forgoing ingredients will be dependent upon the desired end product of the disclosed technology. For example, a hand soap, body wash, shampoo, and facial cleanser can contain different ingredients as well as varying amounts of the same ingredient. The choice and amount of ingredients in formulated compositions of the present technology will vary depending on the product and its function, as is well known to those skilled in the formulation arts.

As defined and used herein, the terms “fatty acid salt”, “fatty acid soap” and “soap” are used interchangeably.

As defined herein, “stable” and “stability” means that no visible phase separation is observed for a period of at least about one week of storage, or at least about 1 month of storage, or at least about 6 months of storage at ambient room temperature (20 to about 25° C.). In another aspect, the products of the disclosed technology show no visible phase separation after about at least four weeks, or at least about 6 weeks, or at least about 8 weeks of storage at 45° C.

While overlapping weight ranges for the various ingredients that make up the cleansing composition will be expressed for various embodiments of the disclosed technology, it should be readily apparent that the specific amount of each component in the composition will be selected from its disclosed range such that the desired amount of each component will be adjusted so that the sum of all components in the cleansing composition totals 100 wt. %.

### Fatty Acid Soap

In one aspect of the disclosed technology the cleansing composition contains at least one the fatty acid salt soap containing from about 8 to about 22 carbon atoms. In another aspect of the disclosed technology the cleansing composition contains at least one fatty acid salt soap containing from about 10 to about 18 carbon atoms. In a further aspect of the disclosed technology the cleansing composition contains at least one fatty acid salt soap containing from about 12 to about 16 carbon atoms. The fatty acids utilized in the soaps can be saturated and unsaturated and can be derived from synthetic sources, as well as from the hydrolysis of fats and natural oils. Exemplary saturated fatty acids include but are not limited to octanoic, decanoic, lauric, myristic, pentadecanoic, palmitic, margaric, steric, isostearic, nonadecanoic, arachidic, behenic, and the like, and mixtures thereof. Exemplary unsaturated fatty acids include but are not limited to myristoleic, palmitoleic, oleic, linoleic, linolenic, and the like, and mixtures thereof. The fatty acids can be derived from animal fat such as tallow, lard, poultry fat or from vegetable sources such as coconut oil, red oil, palm kernel oil, palm oil, cottonseed oil, linseed oil, sunflower seed oil, olive oil, soybean oil, peanut oil, corn oil, safflower oil, sesame oil, rapeseed oil, canola oil, and mixtures thereof.

The soap can be prepared by a variety of well-known means such as by the direct base neutralization of a fatty acid or mixtures thereof or by the saponification of suitable fats and vegetable oils or mixtures thereof with a suitable base. Exemplary bases include ammonium hydroxide, potassium hydroxide, potassium carbonate, sodium hydroxide and alkanol amines such as triethanolamine. Generally, the fat or oil is heated until liquefied and a solution of the desired base is added thereto. Soaps included in a personal care composition utilized in the method of the disclosed technology can be made, for example, by a classic kettle process or modern continuous manufacturing process wherein natural fats and oils such as tallow or coconut oil or their equivalents are saponified with an alkali metal hydroxide using procedures well known to those skilled in the art. Alternatively, soaps can be made by the direct neutralization of free fatty acids such as lauric acid (C₁₂), myristic acid (C₁₄), palmitic acid (C₁₆), steric acid (C₁₈), isostearic (C₁₈), and mixtures thereof, with an alkali metal hydroxide or carbonate. The fatty acid can be pre-neutralized (before addition to the formulation) or can be neutralized in situ during the formulation process.

In one aspect of the disclosed technology, the fatty acid salt soap comprises a fatty acid salt wherein the fatty acid is selected from a mixture of lauric acid, myristic acid, and palmitic acid. In another aspect of the technology, the fatty acid soap is the potassium salt of lauric, myristic and palm itic acids.

The amount of the at least one fatty acid salt soap that is employed in the cleansing compositions of the present technology ranges from about 5 to about 40 wt. %, or from about 8 to about 30 wt. %, or from about 10 to about 25 wt. %, based on the total weight of the composition.

### Amphiphilic Polymer

In one aspect of the disclosed technology, the crosslinked nonionic, amphiphilic polymer component is prepared from monomer components that contain free radically polymerizable monounsaturation. In one aspect, the crosslinked nonionic amphiphilic polymer component is prepared from a polyunsaturated crosslinking monomer. In one aspect, the crosslinked nonionic amphiphilic polymer useful in the practice of the disclosed technology is prepared from a monomer mixture comprising: a) at least one monomer selected from a C₁ to C₅ hydroxyalkyl (meth)acrylate; b) at least one monomer selected from a C₁ to C₅ alkyl (meth)acrylate; c) at least one monomer selected from an associative monomer, a semi-hydrophobic monomer, and mixtures thereof; and d) at least one polyunsaturated crosslinking monomer.

In one aspect, the crosslinked nonionic amphiphilic polymer useful in the practice of the disclosed technology is prepared from a monomer mixture comprising: a) at least one monomer selected from 2-hydroxyethyl methacrylate; b) at least one monomer selected from a ethyl acrylate, butyl acrylate, and mixtures thereof; c) at least one monomer selected from an associative monomer; and mixtures thereof; d) an amphiphilic crosslinking monomer; and e) an amphiphilic additive, wherein said polymerizable monomer mixture containing the amphiphilic additive is free of a protective colloid and/or a polymeric stabilizer. In one embodiment, the monomer mixture is polymerized in a medium containing a protective colloid, a polymeric steric stabilizer and combinations thereof.

The hydroxy(C₁-C₅)alkyl (meth)acrylates can be structurally represented by the following formula:

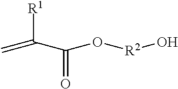

wherein R¹ is hydrogen or methyl and R² is an alkyl moiety containing 1 to 5 carbon atoms, wherein the alkyl moiety optionally can be substituted by one or more methyl groups. Representative monomers include 2-hydroxyethyl(meth)acrylate, 3-hydroxypropyl(meth)acrylate, 4-hydroxybutyl(meth)acrylate, and mixtures thereof.

In one aspect, the amount of the at least one hydroxy(C₁-C₅)alkyl (meth)acrylate monomer(s) present in the monomer mixture utilized to prepare the crosslinked nonionic amphiphilic polymers of the disclosed technology range from about 30 to about 55 wt. %, or from about 35 to about 50 wt. %, or from about 42 to about 48 wt. %, or from about 44 to about 46 wt. %, based on the total weight of monomers in the monomer mixture.

The (C₁-C₅) alkyl (meth)acrylates can be structurally represented by the following formula:

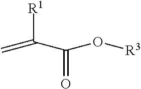

wherein R¹ is hydrogen or methyl and R³ is C₁ to C₅ alkyl. Representative monomers include but are not limited to methyl (meth)acrylate, ethyl (meth)acrylate, n-butyl (meth)acrylate, sec-butyl (meth)acrylate, and iso-butyl (meth)acrylate, and mixtures thereof.

In one aspect, the amount of the at least one C₁ to C₅ alkyl ester of (meth)acrylic acid in the monomer mixture ranges from about 10 to about 55 wt. %, or from about 12 to about 45 wt. %, or from about 15 to about 40 wt. %, or from about 20 to about 35 wt. %, or from about 25 to about 30 wt. %, based on the total weight of monomers in the monomer mixture.

The associative monomer has an ethylenically unsaturated end group portion (i) for addition polymerization with the other monomers in the monomer mixture; a polyoxyalkylene mid-section portion (ii) for imparting selective hydrophilic and/or hydrophobic properties to the product polymer, and a hydrophobic end group portion (iii) for providing selective hydrophobic properties to the polymer.

The portion (i) supplying the ethylenically unsaturated end group can be a residue derived from an α,β-ethylenically unsaturated monocarboxylic acid. Alternatively, portion (i) of the associative monomer can be a residue derived from an allyl ether or vinyl ether; a nonionic vinyl-substituted urethane monomer, such as disclosed in U.S. Reissue Pat. No. 33,156 or U.S. Pat. No. 5,294,692; or a vinyl-substituted urea reaction product, such as disclosed in U.S. Pat. No. 5,011,978; the relevant disclosures of each are incorporated herein by reference.

The mid-section portion (ii) is a polyoxyalkylene segment of about 2 to about 150, or from about 10 to about 120, or from about 15 to about 60 of repeating C₂-C₄ alkylene oxide units. The mid-section portion (ii) includes polyoxyethylene, polyoxypropylene, and polyoxybutylene segments, and combinations thereof comprising from about 2 to about 150, or from about 5 to about 120, or from about 10 to about 60 of ethylene, propylene and/or butylene oxide units, arranged in random or block sequences of ethylene oxide, propylene oxide and/or butylene oxide units.

The hydrophobic end group portion (iii) of the associative monomer is a hydrocarbon moiety belonging to one of the following hydrocarbon classes: a C₈-C₃₀ linear alkyl, a C₈-C₃₀ branched alkyl, a C₈-C₃₀ carbocyclic alkyl, a C₂-C₃₀ alkyl substituted phenyl, an aralkyl substituted phenyl, and aryl substituted C₁-C₁₀ alkyl groups.

Examples of C₈-C₃₀ linear and branched alkyl groups include, without limitation, alkyl groups derived from hydrogenated peanut oil, soybean oil and canola oil (all predominately C₁₈), hydrogenated tallow oil (C₁₆-C₁₈), and the like; and hydrogenated C₁₀-C₃₀ terpenols, such as hydrogenated geraniol (branched C₁₀), hydrogenated farnesol (branched C₁₅), hydrogenated phytol (branched C₂₀), and the like. Non-limiting examples include capryl (C₈), iso-octyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and the like.

Suitable C₈-C₃₀ carbocylic alkyl groups include, without being limited thereto, groups derived from sterols from animal sources, such as cholesterol, lanosterol, 7-dehydrocholesterol, and the like; from vegetable sources, such as phytosterol, stigmasterol, campesterol, and the like; and from yeast sources, such as ergosterol, mycosterol, and the like. Other carbocyclic alkyl hydrophobic end groups useful in the disclosed technology include, without being limited thereto, cyclooctyl, cyclododecyl, adamantyl, decahydronaphthyl, and groups derived from natural carbocyclic materials, such as pinene, hydrogenated retinol, camphor, isobornyl alcohol, and the like.

Non-limiting examples of suitable C₂-C₃₀ alkyl substituted phenyl groups include octylphenyl, nonylphenyl, decylphenyl, dodecylphenyl, hexadecylphenyl, octadecylphenyl, isooctylphenyl, sec-butylphenyl, and the like.

Examples of aryl substituted phenyl groups (e.g., residues of the corresponding phenol) include, without limitation, di- and tri-styryl and di- and tri-cumyl phenyl groups.

Non-limiting examples of suitable aryl substituted C₁-C₁₀ alkyl groups include benzyl, cumyl, phenylethyl, phenyl propyl, phenylbutyl, propyl-2-phenylethy and 3-(4-methylphenyl)propyl.

In one aspect, exemplary associative monomers include those represented by formulas below:

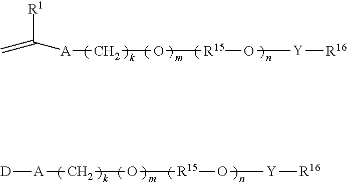

wherein R¹ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150, or from about 10 to about 120, or from about 15 to about 60; Y is —R¹⁵O—, —R¹⁵NH—, —C(O)—, —C(O)NH—, —R¹⁵NHC(O)NH—, or —C(O)NHC(O)—; R¹⁶ is C₈-C₃₀ linear alkyl, a C₈-C₃₀ branched alkyl, a C₈-C₃₀ carbocyclic alkyl, a C₂-C₃₀ alkyl substituted phenyl, an aralkyl substituted phenyl, and aryl substituted C₁-C₁₀ alkyl groups.; wherein the R¹⁶ alkyl group(s), aryl group(s), phenyl group(s) optionally contains one or more substituents selected from a hydroxyl group, a C₁-C₅ alkoxyl group, benzyl group phenylethyl group, and a halogen group.

In one aspect, the hydrophobically modified associative monomer is an alkoxylated (meth)acrylate containing a hydrophobic group containing 8 to 30 carbon atoms represented by the following formula:

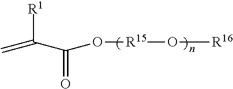

wherein R¹ is hydrogen or methyl; R¹⁵ is a divalent alkylene moiety independently selected from C₂H₄, C₃H₆, and C₄H₈, and n represents an integer ranging from about 2 to about 150, or from about 5 to about 120, or from about 10 to about 60; R¹⁶ is C₈-C₃₀ linear alkyl, a C₈-C₃₀ branched alkyl, a C₈-C₃₀ carbocyclic alkyl, a C₂-C₃₀ alkyl substituted phenyl, an aralkyl substituted phenyl, and aryl substituted C₁-C₁₀ alkyl groups.; wherein the R¹⁶ alkyl group(s), aryl group(s), phenyl group(s) optionally contains one or more substituents selected from a hydroxyl group, a C₁-C₅ alkoxyl group, benzyl group phenylethyl group, and a halogen group.

Representative associative monomers under include lauryl polyethoxylated methacrylate (LEM), cetyl polyethoxylated methacrylate (CEM), cetearyl polyethoxylated methacrylate (CSEM), stearyl polyethoxylated (meth)acrylate, arachidyl polyethoxylated (meth)acrylate, behenyl polyethoxylated methacrylate (BEM), cerotyl polyethoxylated (meth)acrylate, montanyl polyethoxylated (meth)acrylate, melissyl polyethoxylated (meth)acrylate, phenyl polyethoxylated (meth)acrylate, nonylphenyl polyethoxylated (meth)acrylate, ω-tristyrylphenyl polyoxyethylene methacrylate, where the polyethoxylated portion of the monomer contains about 2 to about 150 ethylene oxide units in one aspect, from about 5 to about 120 in another aspect, from about 10 to about 60 in still another aspect, from 10 to 40 in a further aspect, and from 15 to 30 in a still further aspect; octyloxy polyethyleneglycol (8) polypropyleneglycol (6) (meth)acrylate, phenoxy polyethylene glycol (6) polypropylene glycol (6) (meth)acrylate, and nonylphenoxy polyethylene glycol polypropylene glycol (meth)acrylate.

The associative monomers can be prepared by any method known in the art. See, for example, U.S. Pat. No. 4,421,902 to Chang et al.; U.S. Pat. No. 4,384,096 to Sonnabend; U.S. Pat. No. 4,514,552 to Shay et al.; U.S. Pat. No. 4,600,761 to Ruffner et al.; U.S. Pat. No. 4,616,074 to Ruffner; U.S. Pat. No. 5,294,692 to Barron et al.; U.S. Pat. No. 5,292,843 to Jenkins et al.; U.S. Pat. No. 5,770,760 to Robinson; and U.S. Pat. No. 5,412,142 to Wilkerson, III et al.; the pertinent disclosures of which are incorporated herein by reference.

The semi-hydrophobic monomers of the disclosed technology are structurally similar to the associative monomer described above but have a substantially non-hydrophobic end group portion. The semi-hydrophobic monomer has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer and a semi-hydrophobic end group portion (iii). The unsaturated end group portion (i) supplying the vinyl or other ethylenically unsaturated end group for addition polymerization is preferably derived from an a,3-ethylenically unsaturated mono carboxylic acid. Alternatively, the polymerizable end group portion (i) can be derived from an allyl ether residue, a vinyl ether residue or a residue of a nonionic urethane monomer.

The polyoxyalkylene mid-section (ii) specifically comprises a polyoxyalkylene segment, which is substantially similar to the polyoxyalkylene portion of the associative monomers described above. In one aspect, the polyoxyalkylene portions (ii) include polyoxyethylene, polyoxypropylene, and/or polyoxybutylene units comprising from about 2 to about 150 in one aspect, from about 5 to about 120 in another aspect, and from about 10 to about 60 in a further aspect of ethylene oxide, propylene oxide, and/or butylene oxide units, arranged in random or blocky sequences.

The semi-hydrophobic end group portion (iii) is a substantially non-hydrophobic end group selected from hydrogen or a moiety containing 1 to 4 carbon atoms. Exemplary carbon atom containing semi-hydrophobic end groups include methyl, ethyl, propyl and butyl moieties.

In one aspect, the semi-hydrophobic monomer can be represented by the following formulas:

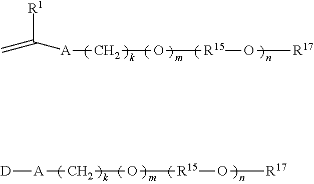

wherein R¹ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer ranging from about 2 to about 150, or from about 5 to about 120, or from about 10 to about 60 in a further aspect; R¹⁷ is selected from hydrogen and a linear or branched C₁-C₄ alkyl group (e.g., methyl, ethyl, propyl, iso-propyl, butyl, iso-butyl, and tert-butyl); and D represents a vinyl or an allyl moiety.

In one aspect, the semi-hydrophobic monomer can be represented by the following formulas:

CH₂═C(R¹)C(O)O—(C₂H₄O)ₐ(C₃H₆O)_{b}—H

CH₂═C(R¹)C(O)O—(C₂H₄O)ₐ(C₃H₆O)_{b}—CH₃

wherein R¹ is hydrogen or methyl, and “a” is an integer ranging from 0 or 2 to about 120, or from about 5 to about 45, or from about 10 to about, and “b” is an integer ranging from about 0 or 2 to about 120, or from about 5 to about 45, or from about 10 to about 25, subject to the proviso that “a” and “b” cannot be 0 at the same time.

Examples of semi-hydrophobic monomers include polyethyleneglycol methacrylate available under the product names Blemmer® PE-90 (R¹=methyl, a=2, b=0), PE-200 (R¹=methyl, a=4.5, b=0), and PE-350 (R¹=methyl a=8, b=0,); polypropylene glycol methacrylate available under the product names Blemmer® PP-1000 (R¹=methyl, b=4-6, a=0), PP-500 (R¹=methyl, a=0, b=9), PP-800 (R¹=methyl, a=0, b=13); polyethyleneglycol polypropylene glycol methacrylate available under the product names Blemmer® 50PEP-300 (R¹=methyl, a=3.5, b=2.5), 70PEP-350B (R¹=methyl, a=5, b=2); polyethyleneglycol acrylate available under the product names Blemmer® AE-90 (R¹=hydrogen, a=2, b=0), AE-200 (R¹=hydrogen, a=2, b=4.5), AE-400 (R¹=hydrogen, a=10, b=0); polypropyleneglycol acrylate available under the product names Blemmer® AP-150 (R¹=hydrogen, a=0, b=3), AP-400(R¹=hydrogen, a=0, b=6), AP-550 (R¹=hydrogen, a=0, b=9). Blemmer® is a trademark of NOF Corporation, Tokyo, Japan.

Additional examples of semi-hydrophobic monomers include methoxypolyethyleneglycol methacrylate available under the product names Visiomer® MPEG 750 MA W (R¹=methyl, a=17, b=0), MPEG 1005 MA W (R¹=methyl, a=22, b=0), MPEG 2005 MA W (R¹=methyl, a=45, b=0), and MPEG 5005 MA W (R¹=methyl, a=113, b=0) from Evonik ROhm GmbH, Darmstadt, Germany); Bisomer® MPEG 350 MA (R¹=methyl, a=8, b=0), and MPEG 550 MA (R¹=methyl, a=12, b=0) from GEO Specialty Chemicals, Ambler PA; Blemmer® PME-100 (R¹=methyl, a=2, b=0), PME-200 (R¹=methyl, a=4, b=0), PME400 (R¹=methyl, a=9, b=0), PME-1000 (R¹=methyl, a=23, b=0), PME-4000 (R¹=methyl, a=90, b=0).

In one aspect, the semi-hydrophobic monomer can be represented by the following formulas:

CH₂═CH—O—(CH₂)_{d}—O—(C₃H₆O)ₑ—(C₂H₄O)_{f}—H

CH₂═CH—CH₂—O—(C₃H₆O)_{g}—(C₂H₄O)ₕ—H

wherein d is an integer of 2, 3, or 4; e is an integer ranging from about 1 to about 10, or from about 2 to about 8, or from about 3 to about 7; f is an integer ranging from about 5 to about 50, or from about 8 to about 40, or from about 10 to about 30 in a further aspect; g is an integer ranging from 1 to about 10, or from about 2 to about 8, or from about 3 to about 7; and h is an integer ranging from about 5 to about 50, or from about 8 to about 40; e, f, g, and h can be 0 subject to the proviso that e and f cannot be 0 at the same time, and g and h cannot be 0 at the same time.

Semi-hydrophobic monomers are commercially available under the trade names Emulsogen® R109, R208, R307, RAL109, RAL208, and RAL307 sold by Clariant Corporation; BX-AA-E5P5 sold by Bimax, Inc.; and combinations thereof. EMULSOGEN® R109 is a randomly ethoxylated/propoxylated 1,4-butanediol vinyl ether having the empirical formula CH₂═CH—O(CH₂)₄O(C₃H₆O)₄(C₂H₄O)₁₀H; Emulsogen® R208 is a randomly ethoxylated/propoxylated 1,4-butanediol vinyl ether having the empirical formula CH₂═CH—O(CH₂)₄O(C₃H₆O)₄(C₂H₄O)₂₀H; Emulsogen® R307 is a randomly ethoxylated/propoxylated 1,4-butanediol vinyl ether having the empirical formula CH₂═CH—O(CH₂)₄O(C₃H₆O)₄(C₂H₄O)₃₀H; Emulsogen® RAL109 is a randomly ethoxylated/propoxylated allyl ether having the empirical formula CH₂═CHCH₂O(C₃H₆O)₄(C₂H₄O)₁₀H; Emulsogen® RAL208 is a randomly ethoxylated/propoxylated allyl ether having the empirical formula CH₂═CHCH₂O(C₃H₆O)₄(C₂H₄O)₂₀H; Emulsogen® RAL307 is a randomly ethoxylated/propoxylated allyl ether having the empirical formula CH₂═CHCH₂O(C₃H₆O)₄(C₂H₄O)₃₀H; and BX-AA-E5P5 is a randomly ethoxylated/propoxylated allyl ether having the empirical formula CH₂═CHCH₂O(C₃H₆O)₅(C₂H₄O)₅H.

In the associative and semi-hydrophobic monomers of the disclosed technology, the polyoxyalkylene mid-section portion contained in these monomers can be utilized to tailor the hydrophilicity and/or hydrophobicity of the polymers in which they are included. For example, mid-section portions rich in ethylene oxide moieties are more hydrophilic while mid-section portions rich in propylene oxide moieties are more hydrophobic. By adjusting the relative amounts of ethylene oxide to propylene oxide moieties present in these monomers the hydrophilic and hydrophobic properties of the polymers in which these monomers are included can be tailored as desired.

The amount of associative and/or semi-hydrophobic monomer utilized in the preparation of the crosslinked nonionic, amphiphilic polymer component of the disclosed technology can vary widely and depends, among other things, on the final rheological and aesthetic properties desired in the polymer. When utilized, the one or more monomers selected from the associative and/or semi-hydrophobic monomers disclosed above can be utilized in amounts ranging from about 0 or 1 to about 20 wt. %, or from about 0.5% to about 18%, or from about 1%, 2%, 3%, 4%, 5%, 6%, 7%, 8%, 9% 10% to about 15 wt. % (based on the total weight of the monounsaturated monomers in the monomer mixture to be polymerized) is utilized to prepare the polymer.

### Ionizable Monomer

In one aspect of the disclosed technology, the crosslinked nonionic amphiphilic polymer compositions of the disclosed technology can be polymerized from a monomer mixture including from about 0 to about 15.0 wt. %, or from about 0.1 to about 15 wt. %, or from about 0.5 to about 10 wt. %, or from about 1 to about 8 wt. %, or from about 2 or 3 to about 5 wt. of an ionizable and/or ionized monomer, based on the weight of the total monomers, so long as the rheological properties or other desirable properties of the composition are not deleteriously affected.

In another aspect, the crosslinked nonionic amphiphilic polymer compositions of the disclosed technology can be polymerized from a monomer mixture comprising less than 3 wt. %, or less than 1 wt. %, or less than 0.5 wt. %, or less than 0.1 wt. %, or less than 0.05 wt. % of an ionizable and/or an ionized moiety, based on the weight of the total monomers in the polymerizable monomer mixture.

Ionizable monomers include monomers having a base neutralizable moiety and monomers having an acid neutralizable moiety. Base neutralizable monomers include olefinically unsaturated monocarboxylic and dicarboxylic acids and their salts containing 3 to 5 carbon atoms and anhydrides thereof. Examples include (meth)acrylic acid, itaconic acid, maleic acid, maleic anhydride, and combinations thereof. Other acidic monomers include styrenesulfonic acid, 2-acrylamido-2-methylpropane sulfonic acid (AMPS® monomer), vinylsulfonic acid, vinylphosphonic acid, allylsulfonic acid, methallylsulfonic acid; and salts thereof.

Acid neutralizable monomers include olefinically unsaturated monomers which contain a basic nitrogen atom capable of forming a salt or a quaternized moiety upon the addition of an acid. For example, these monomers include vinylpyridine, vinylpiperidine, vinylimidazole, vinylmethylimidazole, dimethylaminomethyl (meth)acrylate, dimethylaminoethyl (meth)acrylate, diethylaminomethyl (meth)acrylate and methacrylate, dimethylaminoneopentyl (meth)acrylate, dimethylaminopropyl (meth)acrylate, and diethylaminoethyl (meth)acrylate.

### Crosslinking Monomer

In one aspect, the crosslinked nonionic amphiphilic polymer of the disclosed technology is crosslinked by a conventional polyunsaturated compound. A conventional polyunsaturated compound (conventional crosslinker) is defined herein to be of a relatively low molecular weight (less than 300 Daltons) and contains an average of at least 2 polymerizable unsaturated moieties. In another aspect, the conventional crosslinking agent contains an average of at least 3 unsaturated moieties. Exemplary conventional crosslinkers include di(meth)acrylate compounds such as ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, 2,2′-bis(4-(acryloxy-propyloxyphenyl)propane, and 2,2′-bis(4-(acryloxydiethoxy-phenyl)propane; tri(meth)acrylate compounds such as, trimethylolpropane tri(meth)acrylate, trimethylolethane tri(meth)acrylate, and tetramethylolmethane tri(meth)acrylate; tetra(meth)acrylate compounds such as ditrimethylolpropane tetra(meth)acrylate, tetramethylolmethane tetra(meth)acrylate, and pentaerythritol tetra(meth)acrylate; hexa(meth)acrylate compounds such as dipentaerythritol hexa(meth)acrylate; allyl compounds such as allyl (meth)acrylate, diallylphthalate, diallyl itaconate, diallyl fumarate, and diallyl maleate; polyallyl ethers of sucrose having from 2 to 8 allyl groups per molecule, polyallyl ethers of pentaerythritol such as pentaerythritol diallyl ether, pentaerythritol triallyl ether, and pentaerythritol tetraallyl ether, and combinations thereof; polyallyl ethers of trimethylolpropane such as trimethylolpropane diallyl ether, trimethylolpropane triallyl ether, and combinations thereof. Other suitable polyunsaturated compounds include divinyl glycol, divinyl benzene, and methylenebisacrylamide.

In another aspect, suitable conventional crosslinkers can be synthesized via an esterification reaction of a polyol made from ethylene oxide or propylene oxide or combinations thereof with unsaturated anhydride such as maleic anhydride, citraconic anhydride, itaconic anhydride, or an addition reaction with unsaturated isocyanate such as 3-isopropenyl-α-α-dimethylbenzene isocyanate.

Mixtures of two or more of the foregoing conventional crosslinkers can be utilized to crosslink the nonionic amphiphilic polymers. In one aspect, the mixture of conventional crosslinking monomer contains an average of 2 unsaturated moieties. In another aspect, the mixture of conventional crosslinking agents contains an average of 2.5 unsaturated moieties. In still another aspect, the mixture of conventional crosslinking agents contains an average of about 3 unsaturated moieties. In a further aspect, the mixture of conventional crosslinking agents contains an average of about 3.5 unsaturated moieties.

In one aspect, the conventional crosslinking agent component can be used in an amount ranging from about 0.01 to about 0.5 parts by wt., or from about 0.05 to about 0.4 parts by wt., or from about 0.1 to about 0.3 parts by wt., based on 100 parts by wt. of the monounsaturated monomers utilized to prepare the nonionic, amphiphilic polymers of the disclosed technology.

In one aspect, the conventional crosslinking agent contains an average of about 3 unsaturated moieties and can be used in an amount ranging from about 0.01 to about 0.3 parts by wt. in one aspect, from about 0.02 to about 0.25 parts by wt. in another aspect, from about 0.05 to about 0.2 parts by wt. in a further aspect, and from about 0.075 to about 0.175 parts by wt. in a still further aspect, and from about 0.1 to about 0.15 parts by wt. in another aspect, based on 100 parts by wt. of the monounsaturated monomers utilized to prepare the nonionic, amphiphilic polymers of the disclosed technology.

In one aspect, the conventional crosslinking agent is selected from trimethylolpropane tri(meth)acrylate, trimethylolethane tri(meth)acrylate, tetramethylolmethane tri(meth)acrylate, pentaerythritol triallylether and polyallyl ethers of sucrose having 3 allyl groups per molecule.

In one aspect of the disclosed technology, the crosslinking monomer is an amphiphilic crosslinking agent. The amphiphilic crosslinking agent is utilized to polymerize covalent crosslinks into the amphiphilic polymer backbone. In some instances, conventional crosslinking agents can affect the volume expansion or swelling of micro-gel particles in fluids containing surfactants. For example, a high level of conventional crosslinking agent could provide a high yield stress but the limited expansion of the micro-gels would result in undesirably high polymer use levels and low optical clarity. On the other hand, a low level of conventional crosslinking agents could give high optical clarity but low yield stress. It is desirable that polymeric micro-gels allow maximum swelling while maintaining a desirable yield stress, and it has been found that the use of amphiphilic crosslinking agents in place of, or in conjunction with conventional crosslinking agents can provide these benefits. In addition, it has been found that the amphiphilic crosslinking agent can be easily reacted into the amphiphilic polymer. Often, certain processing techniques, such as staging, can be required with conventional crosslinking agents to achieve the proper balance of optical clarity and yield stress. In contrast, it has been found that amphiphilic crosslinking agents can simply be added in a single stage with the monomer mixture during preparation.

In one aspect, exemplary amphiphilic crosslinking agents suitable for use with the present technology can include, but not be limited to, compounds such as those disclosed in US 2013/0047892 (published Feb. 28, 2013 to Palmer, Jr. et al.), represented by the following formulas:

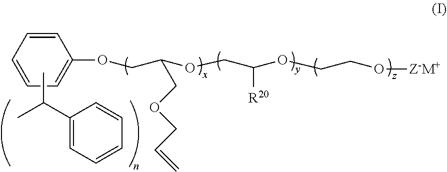

where R²⁰═CH₃, CH₂CH₃, C₆H₅, or C₁₄H₂₉; n=1, 2, or 3; x is 2-10, y is 0-200, z is 4-200, from about 5 to 60 in another aspect, and from about 5 to 40 in a further aspect; Z can be either SO₃⁻ or PO₃²⁻, and M⁺ is Na⁺, K⁺, NH₄⁺, or an alkanolamine such as, for example, monoethanolamine, diethanolamine, and triethanolamine;

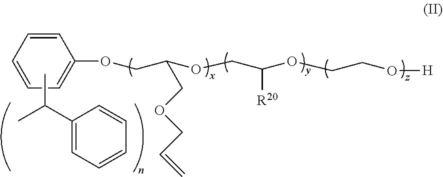

where R²⁰═CH₃, CH₂CH₃, C₆H₅, or C₁₄H₂₉; n=1, 2, 3; x is 2-10, y is 0-200, z is 4-200, from about 5 to 60 in another aspect, and from about 5 to 40 in a further aspect;

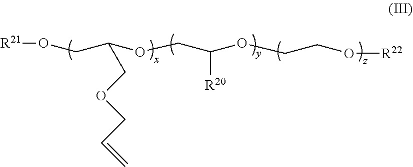

where R²¹ is a C₁₀₋₂₄ alkyl, alkaryl, alkenyl, or cycloalkyl, R²⁰═CH₃, CH₂CH₃, C₆H₅, or C₁₄H₂₉; x is 2-10, y is 0-200, z is 4-200, from about 5 to 60 in another aspect, and from about 5 to 40 in a further aspect; and R²² is H or Z⁻M³⁰ Z can be either SO₃⁻ or PO₃²⁻, and M⁺ is Na⁺, K⁺, NH₄⁺, or an alkanolamine such as, for example, monoethanolamine, diethanolamine, and triethanolamine.

In one aspect, the amphiphilic crosslinking agent is selected from compounds of formulas (IV) or (V) below:

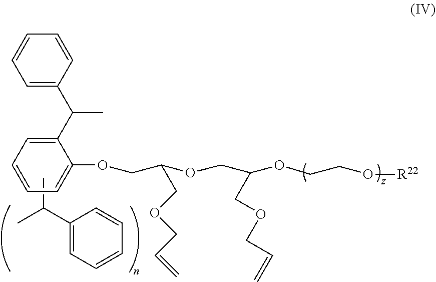

where n is 1 or 2; z is 4 to 40 in one aspect, 5 to 38 in another aspect, and 10 to 20 in a further aspect; and R²² is H, SO₃⁻M⁺ or PO₃⁻² M⁺, and M is selected from Na, K, and NH₄;

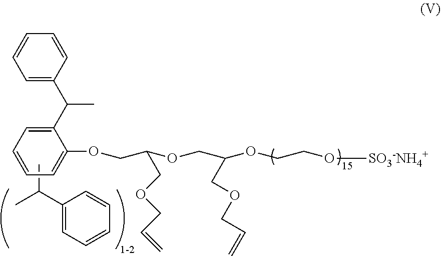

The foregoing amphiphilic crosslinking agents conforming to formulas (I), (II), (III), (IV) and (V) are disclosed in U.S. Patent Application Publication No. US 2014/0114006, the disclosure of which is herein incorporated by reference, and are commercially available under the E-Sperse™ RS Series trade name (e.g., product designations RS-1617, RS-1618, RS-1684) from Ethox Chemicals, LLC.

The amount of polyunsaturated amphiphilic crosslinking monomer utilized to crosslink the polymers of the disclosed technology ranges from about 0.1 to about 5 parts by weight or from about 0.1, 0.2, 0.3, 0.4, 0.5, 0.6, 0.7, 0.8, 0.9 or 1 part to about 5 parts by weight (based on 100 parts by wt. of the monounsaturated monomers utilized to prepare the polymer).

In aspects of the disclosed technology when the nonionic amphiphilic polymer is crosslinked with a combination of a conventional crosslinking agent and an amphiphilic crosslinking agent, the conventional crosslinking agent and amphiphilic crosslinking agent can be used in a total amount ranging from about 0.01 to about 1 parts by wt., or from about 0.05 to about 0.75 parts by wt., or from about 0.1 to about 0.5 parts by wt. in a further aspect, based on 100 parts by wt. of the monounsaturated monomers utilized in the monomer mixture to prepare the nonionic amphiphilic polymers of the disclosed technology.

In one aspect, the combination of the conventional crosslinking agent and amphiphilic crosslinking agent can include conventional crosslinking agents selected from selected from trimethylolpropane tri(meth)acrylate, trimethylolethane tri(meth)acrylate, tetramethylolmethane tri(meth)acrylate, pentaerythritol triallylether and polyallyl ethers of sucrose having 3 allyl groups per molecule, and combinations thereof, and amphiphilic crosslinking agents selected from compounds of formula (III), (V), and combinations thereof.

### Amphiphilic Polymer Synthesis

The crosslinked, nonionic, amphiphilic polymer of the disclosed technology can be made using conventional free-radical emulsion polymerization techniques. The polymerization processes are carried out in the absence of oxygen under an inert atmosphere such as nitrogen. The polymerization can be carried out in a suitable solvent system such as water. Minor amounts of a hydrocarbon solvent, organic solvent, as well as mixtures thereof can be employed. To facilitate emulsification of the monomer mixture, the emulsion polymerization is carried out in the presence of at least one stabilizing surfactant. The polymerization reactions are initiated by any means which results in the generation of a suitable free-radical. Thermally derived radicals, in which the radical species is generated from thermal, homolytic dissociation of peroxides, hydroperoxides, persulfates, percarbonates, peroxyesters, hydrogen peroxide and azo compounds can be utilized. The initiators can be water soluble or water insoluble depending on the solvent system employed for the polymerization reaction.

The initiator compounds can be utilized in an amount of up to 30 wt. % in one aspect, 0.01 to 10 wt. % in another aspect, and 0.2 to 3 wt. % in a further aspect, based on the total weight of the dry polymer.

Exemplary free radical water soluble initiators include, but are not limited to, inorganic persulfate compounds, such as ammonium persulfate, potassium persulfate, and sodium persulfate; peroxides such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid, and water soluble azo compounds, such as 2,2′-azobis(tert-alkyl) compounds having a water solubilizing substituent on the alkyl group. Exemplary free radical oil soluble compounds include, but are not limited to 2,2′-azobisisobutyronitrile, and the like. The peroxides and peracids can optionally be activated with reducing agents, such as sodium bisulfite, sodium formaldehyde, or ascorbic acid, transition metals, hydrazine, and the like.

In one aspect, azo polymerization catalysts include the Vazo® free-radical polymerization initiators, available from DuPont, such as Vazo® 44 (2,2′-azobis(2-(4,5-dihydroimidazolyl)propane), Vazo® 56 (2,2′-azobis(2-methylpropionamidine) dihydrochloride), Vazo® 67 (2,2′-azobis(2-methylbutyronitrile)), and Vazo® 68 (4,4′-azobis(4-cyanovaleric acid)).

Optionally, the use of known redox initiator systems as polymerization initiators can be employed. Such redox initiator systems include an oxidant (initiator) and a reductant. Suitable oxidants include, for example, hydrogen peroxide, sodium peroxide, potassium peroxide, t-butyl hydroperoxide, t-amyl hydroperoxide, cumene hydroperoxide, sodium perborate, perphosphoric acid and salts thereof, potassium permanganate, and ammonium or alkali metal salts of peroxydisulfuric acid, typically at a level of 0.01% to 3.0% by weight, based on dry polymer weight, are used. Suitable reductants include, for example, alkali metal and ammonium salts of sulfur-containing acids, such as sodium sulfite, bisulfite, thiosulfate, hydrosulfite, sulfide, hydrosulfide or dithionite, formadinesulfinic acid, hydroxymethanesulfonic acid, acetone bisulfite, amines such as ethanolamine, glycolic acid, glyoxylic acid hydrate, ascorbic acid, isoascorbic acid, lactic acid, glyceric acid, malic acid, 2-hydroxy-2-sulfinatoacetic acid, tartaric acid and salts of the preceding acids typically at a level of 0.01% to 3.0% by weight, based on dry polymer weight, is used. In one aspect, combinations of peroxodisulfates with alkali metal or ammonium bisulfites can be used, for example, ammonium peroxodisulfate and ammonium bisulfite. In another aspect, combinations of hydrogen peroxide containing compounds (t-butyl hydroperoxide) as the oxidant with ascorbic or erythorbic acid as the reductant can be utilized. The ratio of peroxide-containing compound to reductant is within the range from 30:1 to 0.05:1.

In one aspect, the polymerization can be carried out the presence of a chain transfer agent. Suitable chain transfer agents include, but are not limited to, thio- and disulfide containing compounds, such as C₁-C₁₈ alkyl mercaptans, such as tert-butyl mercaptan, n-octyl mercaptan, n-dodecyl mercaptan, tert-dodecyl mercaptan hexadecyl mercaptan, dodecyl mercaptan, octadecyl mercaptan; mercaptoalcohols, such as 2-mercaptoethanol, 2-mercaptopropanol; mercaptocarboxylic acids, such as mercaptoacetic acid and 3-mercaptopropionic acid; mercaptocarboxylic acid esters, such as butyl thioglycolate, isooctyl thioglycolate, dodecyl thioglycolate, isooctyl 3-mercaptopropionate, and butyl 3-mercaptopropionate; thioesters; C₁-C₁₈ alkyl disulfides; aryldisulfides; polyfunctional thiols such as trimethylolpropane-tris-(3-mercaptopropionate), pentaerythritol-tetra-(3-mercaptopropionate), pentaerythritol-tetra-(thioglycolate), pentaerythritol-tetra-(thiolactate), dipentaerythritol-hexa-(thioglycolate), and the like; phosphites and hypophosphites; C₁-C₄ aldehydes, such as formaldehyde, acetaldehyde, propionaldehyde; haloalkyl compounds, such as carbon tetrachloride, bromotrichloromethane, and the like; hydroxylammonium salts such as hydroxylammonium sulfate; formic acid; sodium bisulfite; isopropanol; and catalytic chain transfer agents such as, for example, cobalt complexes (e.g., cobalt (II) chelates).

The chain transfer agents are generally used in amounts ranging from 0.1 to 10 wt. %, based on the total weight of the monomers present in the polymerization medium.

The polymerization reaction can be carried out at temperatures ranging from 20 to 200° C., from 50 to 150° C., or from 60 to 100° C.

In emulsion polymerization processes it can be advantageous to stabilize the monomer/polymer droplets or particles by means of surface active auxiliaries. Typically, these are emulsifiers or protective colloids. Emulsifiers used can be anionic, nonionic, cationic or amphoteric. Examples of anionic emulsifiers are alkylbenzenesulfonic acids, sulfonated fatty acids, sulfosuccinates, fatty alcohol sulfates, alkylphenol sulfates and fatty alcohol ether sulfates. Examples of usable nonionic emulsifiers are alkylphenol ethoxylates, primary alcohol ethoxylates, fatty acid ethoxylates, alkanolamide ethoxylates, fatty amine ethoxylates, ethylene oxide/propylene oxide block copolymers and alkylpolyglucosides. Examples of cationic and amphoteric emulsifiers used are quaternized amine alkoxylates, alkylbetaines, alkylamidobetaines and sulfobetaines.

Examples of typical protective colloids are cellulose derivatives, polyethylene glycol, polypropylene glycol, copolymers of ethylene glycol and propylene glycol, polyvinyl acetate, poly(vinyl alcohol), partially hydrolyzed poly(vinyl alcohol), polyvinyl ether, starch and starch derivatives, dextran, polyvinylpyrrolidone, polyvinylpyridine, polyethyleneimine, polyvinylimidazole, polyvinylsuccinimide, polyvinyl-2-methylsuccinimide, polyvinyl-1,3-oxazolid-2-one, polyvinyl-2-methylimidazoline and maleic acid or anhydride copolymers. The emulsifiers or protective colloids are customarily used in concentrations from 0.05 to 20 wt. %, based on the weight of the total monomers.

In one aspect, the emulsion process can be conducted in the absence of a protective colloid. In this aspect, the emulsion process employs an amphiphilic additive. In accordance with one aspect of the present technology the amphiphilic additive is mixed into the polymerizable monomer mixture containing the amphiphilic crosslinking agent before introducing the monomer mixture into the polymerization medium. The monomer mixture (disperse phase) as well as the polymerization medium (continuous phase) is devoid of a protective colloid such as, for example, poly(vinyl alcohol) and poly(vinyl acetate) and/or a polymeric steric stabilizer. Surprisingly, it has been found that by mixing an amphiphilic additive with the polymerizable monomer mixture and removing the protective colloid from the emulsion polymerization medium the clarity and turbidity properties of surfactant compositions containing the resultant polymer product is improved.

The amphiphilic additives of the present technology are nonionic and contain at least one hydrophilic segment and at least two hydrophobic segments.

In one aspect, the amphiphilic additive of the present technology is represented by the formula:

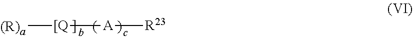

wherein Q represents a polyol residue; A represents a poly(ethylene glycol) residue; R is selected from a saturated and unsaturated C₁₀ to C₂₂ acyl group and a poly(propylene glycol) residue; R²³ is independently selected from H, a saturated and unsaturated C₁₀ to C₂₂ acyl radical and a poly(propylene glycol) residue; a is 0 or 1; b is 0 or 1; and c is a number from 1 to 4; subject to the proviso that when b is 0, a and c are 1, and when b is 1, a is 0 and R²³ is not a poly(propylene glycol) residue.

In one aspect of the disclosed technology, the amphiphilic additive is a polyethoxylated alkyl glucoside ester represented by the formula:

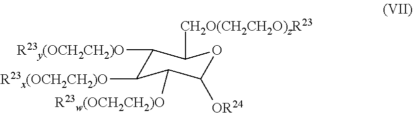

wherein R²³ is independently selected from H and a saturated and unsaturated C₁₀-C₂₂ acyl group; R²⁴ is selected from a C₁-C₁₀ alkyl group; and the sum of w+x+y+z ranges from about 60 to about 150, or from about 80 to about 135, or from about 90 to about 125, or from about 100 to about 120; subject to the proviso that at no more than two of R²³ can be H at the same time.

In one aspect R²³ is an acyl residue of lauric acid, myristic acid, palmitic acid, palmitoleic acid, steric acid, isostearic acid, oleic acid, ricinoleic acid vaccenic acid, linoleic acid (alpha and gamma), arachidic acid, behenic acid, and mixtures thereof and R²⁵ is methyl.

Suitable polyethoxylated alkyl glucoside esters are commercially available under the trade names Glucamate™ LT (INCI Name: PEG-120 Methyl Glucose Trioleate (and) Propylene Glycol (and) Water), Glucamate™ VLT (INCI Name: PEG-120 Methyl Glucose Trioleate (and) Propanediol), and GlucamateTM DOE-120 (INCI Name: PEG-120 Methyl Glucose Dioleate).

In one aspect of the disclosed technology, the amphiphilic additive is selected from a poly(ethylene glycol) diester where poly(ethylene glycol) (PEG) is esterified with a saturated and unsaturated C₁₀ to C₂₂ fatty acid is represented by the formula:

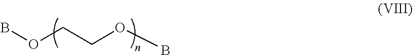

wherein B is independently selected from a saturated and unsaturated C₁₀ to C₂₂ acyl radical; and n ranges from about 10 to about 120, or from about 12 to about 110, or from about 15 to about 100.

In one aspect B is an acyl residue of lauric acid, myristic acid, palmitic acid, palmitoleic acid, steric acid, isostearic acid, oleic acid, ricinoleic acid vaccenic acid, linoleic acid (alpha and gamma), arachidic acid, behenic acid, and mixtures thereof.

Exemplary PEG diesters include but are not limited to the laurate, palmitate, palmitoleate, stearate, isostearate, and oleate diesters of PEG-400, PEG-600, PEG-1000, PEG-2000, and PEG-4000.

In one aspect of the disclosed technology, the amphiphilic additive is a poly(propylene glycol)-block-poly(ethylene glycol)-block-poly(propylene glycol)-block copolymer represented by the formula:

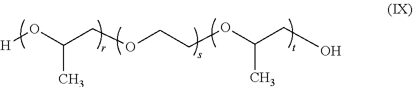

wherein r=t and range from about 5 to about 20, or from about 6 to about 15, or from about 8 to about 14; and s ranges from about 20 to about 30, or from about 21 to about, or from about 23 to about 25.

In one aspect, the poly(propylene glycol)-block-poly(ethylene glycol)-block-poly(propylene glycol)-block copolymer has a number average molecular weight ranging from about 1500 to about 3500 Da.

The poly(propylene glycol)-block-poly(ethylene glycol)-block-poly(propylene glycol)-block copolymer contains from about 35 to about 60, or from about 40 to about 55 wt. %, or from about 45 to about 50 wt. % of poly(ethylene glycol). Suitable poly(propylene glycol)-block-poly(ethylene glycol)-block-poly(propylene glycol)-block copolymers are marketed under the Pluronic™ 10R5 and Pluronic™ 17R4 trade names by BASF Corporation, Florham Park, N.J.

The amount of amphiphilic additive that is mixed with the polymerizable monomer mixture ranges from about 1 to about 15 parts by wt., or from about 2 to about 10 parts by wt., or from about 3 to about 6 parts by wt., based upon 100 parts by wt. of the monounsaturated monomers in the polymerizable monomer mixture utilized to prepare the nonionic, amphiphilic polymers of the disclosed technology.

The emulsion process can be conducted in in a single reactor or in multiple reactors as is well-known in the art. The monomers can be added as a batch mixture or each monomer can be metered into the reactor in a staged process. A typical mixture in emulsion polymerization comprises water, monomer(s), an initiator (usually water-soluble) and an emulsifier. The monomers may be emulsion polymerized in a single-stage, two-stage or multi-stage polymerization process according to well-known methods in the emulsion polymerization art. In a two-stage polymerization process, the first stage monomers are added and polymerized first in the aqueous medium, followed by addition and polymerization of the second stage monomers. The aqueous medium optionally can contain an organic solvent. If utilized, the organic solvent is less than about 5 wt. % of the aqueous medium. Suitable examples of water-miscible organic solvents include, without limitation, esters, alkylene glycol ethers, alkylene glycol ether esters, lower molecular weight aliphatic alcohols, and the like.

To facilitate emulsification of the monomer mixture, the emulsion polymerization is carried out in the presence of at least one stabilizing surfactant. The term “stabilizing surfactant” is used in the context of surfactants employed to facilitate emulsification. In one apect, the emulsion polymerization is carried out in the presence of stabilizing surfactant (active weight basis) ranging in the amount from about 0.2 to about 5 wt. %, or from about 0.5 to about 3 wt. %, or from about 1 to about 2 wt. %, based on the total monomer weight. in the polymerizable mixture. The emulsion polymerization reaction mixture also includes one or more free radical initiators which are present in an amount ranging from about 0.01 to about 3 wt. % based on total monomer weight of the polymerizable monomer mixture. The polymerization can be performed in an aqueous or aqueous alcohol medium. Stabilizing surfactants for facilitating the emulsion polymerization include anionic, nonionic, amphoteric, and cationic surfactants, as well as reactive derivatives thereof, and mixtures thereof. By “reactive derivatives thereof” it is meant surfactants, or mixtures of surfactants, having on average less than one reactive moiety. Most commonly, anionic and nonionic surfactants can be utilized as stabilizing surfactants as well as mixtures thereof.

Suitable anionic surfactants for facilitating emulsion polymerization are well known in the art and include, but are not limited to (C₆-C₁₈) alkyl sulfates, (C₆-C₁₈) alkyl ether sulfates (e.g., sodium lauryl sulfate and sodium laureth sulfate), amino and alkali metal salts of dodecylbenzenesulfonic acid, such as sodium dodecyl benzene sulfonate and dimethylethanolamine dodecylbenzenesulfonate, sodium (C₆-C₁₆) alkyl phenoxy benzene sulfonate, disodium (C₆-C₁₆) alkyl phenoxy benzene sulfonate, disodium (C₆-C₁₆) di-alkyl phenoxy benzene sulfonate, disodium laureth-3 sulfosuccinate, sodium dioctyl sulfosuccinate, sodium di-sec-butyl naphthalene sulfonate, disodium dodecyl diphenyl ether sulfonate, disodium n-octadecyl sulfosuccinate, phosphate esters of branched alcohol ethoxylates, and the like, as well as reactive derivatives thereof.

Nonionic surfactants suitable for facilitating emulsion polymerizations are well known in the polymer art, and include, without limitation, linear or branched C₈-C₃₀ fatty alcohol ethoxylates, such as capryl alcohol ethoxylate, lauryl alcohol ethoxylate, myristyl alcohol ethoxylate, cetyl alcohol ethoxylate, stearyl alcohol ethoxylate, cetearyl alcohol ethoxylate, sterol ethoxylate, oleyl alcohol ethoxylate, and, behenyl alcohol ethoxylate; alkylphenol alkoxylates, such as octylphenol ethoxylates; and polyoxyethylene polyoxypropylene block copolymers, and the like, as well as reactive derivatives thereof. Additional fatty alcohol ethoxylates suitable as non-ionic surfactants are described below. Other useful nonionic surfactants include C₈-C₂₂ fatty acid esters of polyoxyethylene glycol, ethoxylated mono- and diglycerides, sorbitan esters and ethoxylated sorbitan esters, C₈-C₂₂ fatty acid glycol esters, block copolymers of ethylene oxide and propylene oxide, and combinations thereof, as well as reactive derivatives thereof. The number of ethylene oxide units in each of the foregoing ethoxylates can range from 2 and above, or from 2 to about 150.

Optionally, other emulsion polymerization additives and processing aids which are known in the emulsion polymerization art, such as solvents, protective colloids, buffering agents, chelating agents, inorganic electrolytes, biocides, and pH adjusting agents can be included in the polymerization system.

In one aspect, a two-stage emulsion polymerization reaction is utilized to prepare the polymers of the present technology. A mixture of the monounsaturated monomers, the crosslinking agent(s) and the protective colloid or amphiphilic additive is added to a first reactor under inert atmosphere to a solution of emulsifying surfactant (e.g., anionic surfactant) in water. The monomer mixture is devoid of a protective colloid and/or a polymeric steric stabilizer such as poly(vinyl alcohol or poly(vinyl acetate) if the amphiphilic additive is utilized. The contents of the first reactor are agitated to prepare a monomer emulsion (disperse phase). To a second reactor equipped with an agitator, an inert gas inlet, and feed pumps are added under inert atmosphere a desired amount of water and additional anionic surfactant (dispersing medium or continuous phase). The contents of the second reactor are heated with mixing agitation. After the contents of the second reactor reaches a temperature in the range of about 55 to 98° C., a free radical initiator is injected into the aqueous surfactant solution, and the monomer emulsion from the first reactor is gradually metered into the second reactor over a period typically ranging from about one half to about four hours. The reaction temperature is controlled in the range of about 45 to about 95° C. After completion of the monomer addition, an additional quantity of free radical initiator can optionally be added to the second reactor, and the resulting reaction mixture is typically held at a temperature of about 45 to 95° C. for a time sufficient to complete the polymerization reaction to obtain the polymer emulsion.

In one aspect, the crosslinked nonionic amphiphilic polymers of the disclosed technology are selected from an emulsion polymer polymerized from a monomer mixture comprising from about 20 to about 55 wt. % of at least one C₁-C₅ hydroxyalkyl (meth)acrylate; from about 10 to about 50 wt. % of at least one C₁-C₅ alkyl (meth)acrylate; from about 0.1, 1, 5, or 7 to about 20 wt. % of at least one associative and/or a semi-hydrophobic monomer (wherein all monomer weight percentages are based on the total weight of the monounsaturated monomers); and from about 0.01 to about 5 parts by wt., or from about 0.1 to about 3 parts by wt., or from about 0.3 to about 3 parts by wt. of at least one crosslinker (based upon 100 parts by wt. of the monounsaturated monomers utilized in the monomer mixture used to prepare the polymer), wherein the crosslinker is selected from a conventional crosslinking agent, an amphiphilic crosslinking agent, and mixtures thereof.

In one aspect, the crosslinked nonionic amphiphilic polymers of the disclosed technology are selected from an emulsion polymer polymerized from a monomer mixture comprising from about 40 to 50 wt. %, or 42 to 48 wt. %, or 44 to 46 wt. % of hydroxyethyl methacrylate; from about 10 to about 40 wt. %, or 12 to 35 wt. % or 15 to 25 wt. % of ethyl acrylate; from about 10 to about 35 wt. %, or 12 to 30 wt. %, or 15 to 25 wt. % of butyl acrylate; from about 0.5 to about 18 wt. %, or from about 1, 2, 3, 4, 5, 6, 7, 8, 9, 10 to about 15 wt. % of at least one associative monomer (wherein all monomer weight percentages are based on the weight of the total monomers in the polymerizable monomer mixture); and from about 0.01 to about 5 parts by wt. in one aspect, from about 0.1 to about 3 parts by wt. in another aspect, and from about 0.3 to about 3 parts by wt. in a further aspect of at least crosslinker (based on 100 parts by wt. of the monounsaturated monomers in the monomer mixture utilized to prepare the polymer), wherein the crosslinker is selected from a conventional crosslinking agent, an amphiphilic crosslinking agent, and mixtures thereof.

In one aspect, the crosslinked nonionic amphiphilic polymers of the disclosed technology are selected from an emulsion polymer polymerized from a monomer mixture comprising from about 40 to 50 wt. % of hydroxyethyl methacrylate; from about 10 to about 40 wt. % of ethyl acrylate; from about 12 to about 30 wt. % butyl acrylate; from about 5 or 6 to about 15 wt. % of at least one associative monomer selected from from lauryl polyethoxylated (meth)acrylate, cetyl polyethoxylated (meth)acrylate, cetearyl polyethoxylated (meth)acrylate, stearyl polyethoxylated (meth)acrylate, arachidyl polyethoxylated (meth)acrylate, behenyl polyethoxylated (meth)acrylate, cerotyl polyethoxylated (meth)acrylate, montanyl polyethoxylated (meth)acrylate, melissyl polyethoxylated (meth)acrylate, where the polyethoxylated portion of the monomer contains about 2 to about 50 ethylene oxide units (wherein all monomer weight percentages are based on the weight of the total monomers in the polymerizable monomer mixture); and from about 0.01 to about 5 parts by wt. in one aspect, from about 0.1 to about 3 parts by wt. in another aspect, and from about 0.5 to about 1 part by wt. in a further aspect of at least one polyunsaturated crosslinker (based on 100 parts by wt. of the monounsaturated monomers utilized in the polymerizable monomer mixture used to prepare the polymer), wherein the crosslinker is selected from a conventional crosslinking agent, an amphiphilic crosslinking agent, and mixtures thereof.

The amount of the crosslinked nonionic amphiphilic polymer employed in the compositions of the present technology range from about 1 to about 5 wt. %, or from about 1.5 to about 3 wt. %, or from about 2 to about 2.5 wt. % (active solids), based on the weight of the composition.

### Oily Phase

In one aspect, the oily phase component of the present technology is selected from a non-polar hydrocarbon oil, a non-polar silicone oil, and mixtures thereof. In one aspect, the non-polar oils are nonionic lipophilic compounds that are water insoluble and liquid at room temperature (25° C.). The term water insoluble refers to a compound having a solubility in water of less than 1% at spontaneous pH (at atmospheric pressure and 25° C.). In one aspect, the non-polar oils are selected from a hydrocarbon oil, a silicone oil, and mixtures thereof.

In one aspect, non-polar hydrocarbon oils include volatile hydrocarbon oil, non-volatile hydrocarbon oil, and mixtures thereof. Suitable volatile non-polar hydrocarbon oils include linear or branched, optionally cyclic, C₅-C₂₀ lower alkanes. Examples include, but are not limited to pentane, hexane, heptane, decane, undecane, dodecane, tridecane, tetradecane and C₈-C₁₈ isoparaffins, for example, isodecane, isododecane and isohexadecane.

In one aspect, suitable non-polar hydrocarbon oils are the volatile paraffinic hydrocarbons mentioned above which have a molecular weight of 70-225 Daltons, preferably 160 to 190 Daltons and a boiling point range of 30 to 320° C., or 60 to 260° C., and a viscosity of less than about 10 cst. at 25° C. Such paraffinic hydrocarbons are available from EXXON under the Isopars tradename, and from the Permethyl Corporation. Suitable C₁₂ isoparaffins (isododecane) are manufactured by Permethyl Corporation under the trademark Permethyl 99A. A C₁₆ isoparaffin (isohexadecane) that is commercially available under the Permethyl 101A tradename, is also suitable.

Suitable non-volatile, non-polar hydrocarbon oils include linear or branched hydrocarbons containing at least 20 carbon atoms, such as paraffinic hydrocarbons and olefins. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, petrolatum, pentahydrosqualene, squalene, squalane, and mixtures thereof. In one aspect, such hydrocarbons have a molecular weight ranging from about 300 to 1000 Daltons.

In one aspect, the non-polar oil phase can also contain a non-polar linear silicone oil or may consist entirely of such oil. Silicone oils are synthetic polymeric compounds in which the silicon atoms are bonded together via oxygen atoms. In one aspect, the silicone oil is non-volatile and insoluble in the aqueous phase of the cleansing composition. By non-volatile is meant that the silicone has a very low vapor pressure at ambient temperature conditions (e.g., less than 2 mm Hg at 20° C.). The non-volatile silicone conditioning agent has a boiling point above about 250° C., or above about 260° C., or above about 275° C. in a further aspect. Background information on silicones including sections discussing silicone oils, gums, and resins, as well as their manufacture, are found in Encyclopedia of Polymer Science and Engineering, vol. 15, 2d ed., pp 204-308, John Wiley & Sons, Inc. (1989).

The non-volatile silicone oils have a viscosity ranging from about above about 25 to about 1,000,000 mPa·s at 25° C., or from about 100 to about 600,000 mPa·s, or from about 1000 to about 100,000 mPa·s, or from about 2,000 to about 50,000 mPa·s, or from about 4,000 to about 40,000 mPa·s. In one aspect the silicone oils have an average molecular weight below about 200,000 Daltons. The average molecular weight can typically range from about 400 to about 199,000 Daltons, or from about 500 to about 150,000 Daltons, or from about 1,000 to about 100,000 Daltons, or from about 5,000 to about 65,000 Daltons.

In one aspect, silicone oils suitable as non-polar oils are polyorganosiloxane materials selected from polyalkylsiloxanes, polyarylsiloxanes, polyalkylarylsiloxanes, and mixtures thereof. Methyl substituted polyorganosiloxanes are also known as polydimethylsiloxanes (PDMS) or dimethicone (INCI). Dimethicone is available in various chain lengths and with various molecular weights.

The amount of the non-polar hydrocarbon oil and/or non-polar silicone oil that can be employed in the cleansing compositions of the present technology ranges from about 10 to about 45 wt. %, or from about 12 to about 40 wt. %, or from about 15 to about 35 wt. %, or from about 18 to about 30 wt. %, or from about 20 to about 25 wt. %, based on the total weight of the composition.

### Auxiliary Detersive Surfactants

In one aspect, the personal cleansing composition of the present technology can contain an auxiliary synthetic surfactant (syndet) in addition to the fatty acid soap. The syndet is selected from anionic, cationic, amphoteric, and nonionic surfactants, as well as mixtures thereof.

In one aspect of the present technology, suitable anionic surfactants (non-soaps) include but are not limited to alkyl sulfates, alkyl ether sulfates, alkyl sulphonates, alkaryl sulfonates, α-olefin-sulphonates, alkylamide sulphonates, alkarylpolyether sulphates, alkylamidoether sulfates, alkyl monoglyceryl ether sulfates, alkyl monoglyceride sulfates, alkyl monoglyceride sulfonates, alkyl succinates, alkyl sulfosuccinates, alkyl ether sulfosuccinates, alkyl sulfosuccinamates, alkyl am idosulfosuccinates; alkyl sulfoacetates, alkyl phosphates, alkyl ether phosphates, alkyl ether carboxylates, alkyl am idoethercarboxylates, acyl lactylates, alkyl isethionates, acyl isethionates, carboxylate salts and amino acid derived surfactants such as N-alkyl amino acids, N-acyl amino acids, as well as alkyl peptides. Mixtures of these anionic surfactants are also useful.

In one aspect, the cation moiety of the forgoing surfactants is selected from sodium, potassium, magnesium, ammonium, and alkanolammonium ions such as monoethanolammonium, diethanolammonium triethanolammonium ions, as well as monoisopropylammonium, diisopropylammonium and triisopropylammonium ions. In one embodiment, the alkyl and acyl groups of the foregoing surfactants contain from about 6 to about 24 carbon atoms in one aspect, from 8 to 22 carbon atoms in another aspect and from about 12 to 18 carbon atoms in a further aspect and may be unsaturated. The aryl groups in the surfactants are selected from phenyl or benzyl. The ether containing surfactants set forth above can contain from 1 to 10 ethylene oxide and/or propylene oxide units per surfactant molecule in one aspect, and from 1 to 3 ethylene oxide units per surfactant molecule in another aspect.

Examples of suitable anionic surfactants include the sodium, potassium, lithium, magnesium, and ammonium salts of laureth sulfate, trideceth sulfate, myreth sulfate, C₁₂-C₁₃ pareth sulfate, C₁₂-C₁₄ pareth sulfate, and C₁₂-C₁₅ pareth sulfate, ethoxylated with 1, 2, and 3 moles of ethylene oxide; the sodium potassium, lithium, magnesium, ammonium, and triethanolammonium salts of lauryl sulfate, coco sulfate, tridecyl sulfate, myristyl sulfate, cetyl sulfate, cetearyl sulfate, stearyl sulfate, oleyl sulfate, and tallow sulfate, disodium lauryl sulfosuccinate, disodium laureth sulfosuccinate, sodium cocoyl isethionate, sodium lauroyl isethionate, sodium lauroyl methyl isethionate, sodium C₁₂-C₁₄ olefin sulfonate, sodium laureth-6 carboxylate, sodium dodecylbenzene sulfonate, and triethanolamine monolauryl phosphate.

In one aspect, the amino acid surfactants are selected from a N-acyl amino acid of the formula:

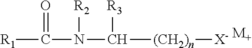

wherein R₁ is a saturated or unsaturated, straight or branched alkyl chain containing 7 to 17 carbon atoms, R₂ is H or a methyl group, R₃ is H, COO⁻M⁺, CH₂COO⁻M⁺ or COOH, n is 0 to 2, X is COO⁻ or SO₃⁻ and M independently represents H, sodium, potassium, ammonium or triethanolammonium.

In one aspect, the N-acyl amino acid surfactants represented by the formula immediately above are derived from taurates, glutamates, alanine, alaninates, sacosinates, aspartates, glycinates, and mixtures thereof.

Representative taurate surfactants conform to the formula:

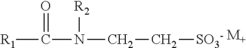

wherein R₁ is a saturated or unsaturated, straight or branched alkyl chain containing 7 to 17 carbon atoms in one aspect and 9 to 13 carbon atoms in another aspect, R2 is H or methyl, and M is H, sodium, potassium, ammonium or triethanolammonium.

Non-limiting examples of taurate surfactants are potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, and mixtures thereof.

Representative glutamate surfactants conform to the formula:

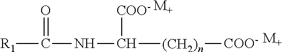

wherein R₁ is a saturated or unsaturated, straight or branched alkyl chain containing 7 to 17 carbon atoms in one aspect and 9 to 13 carbon atoms in another aspect, n is 0 to 2, and M independently is H, sodium, potassium, ammonium or triethanolammonium.

Non-limiting examples of glutamate surfactants are di-potassium capryloyl glutamate, di-potassium undecylenoyl glutamate, di-sodium capryloyl glutamate, di-sodium cocoyl glutamate, di-sodium lauroyl glutamate, di-sodium stearoyl glutamate, di-sodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, and mixtures thereof.

Representative alanine and alaninate surfactants conform to the formula:

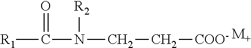

wherein R₁ is a saturated or unsaturated, straight or branched alkyl chain containing 7 to 17 carbon atoms in one aspect and 9 to 13 carbon atoms in another aspect, R₂ is H or methyl, and M is H, sodium, potassium, ammonium or triethanolammonium.

Non-limiting examples of alanine and alaninate surfactants are cocoyl methyl β-alanine, lauroyl β-alanine, lauroyl methyl β-alanine, myristoyl β-alanine, potassium lauroyl methyl β-alanine, sodium cocoyl alaninate, sodium cocoyl methyl β-alanine, sodium myristoyl methyl β-alanine, and mixtures thereof.

Representative glycinate surfactants conform to the formula:

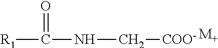

wherein R₁ is a saturated or unsaturated, straight or branched alkyl chain containing 7 to 17 carbon atoms in one aspect and 9 to 13 carbon atoms in another aspect, and M is H, sodium, potassium, ammonium or triethanolammonium.

Non-limiting examples of glycinate surfactants are sodium palmitoyl glycinate, sodium lauroyl glycinate, sodium cocoyl glycinate, sodium myristoyl glycinate, potassium lauroyl glycinate, potassium cocoyl glycinate, sodium stearoyl glycinate, and mixtures thereof.

Representative sarcosinate surfactants conform to the formula:

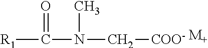

wherein R₁ is a saturated or unsaturated, straight or branched alkyl chain containing 7 to 17 carbon atoms in one aspect and 9 to 13 carbon atoms in another aspect, and M is H, sodium, potassium, ammonium or triethanolamine.

Non-limiting examples of sarcosinate surfactants are potassium lauroyl sarcosinate, potassium cocoyl sarcosinate, sodium cocoyl sarcosinate, sodium lauroyl sarcosinate, sodium myristoyl sarcosinate, sodium palmitoyl sarcosinate, and mixtures thereof.

Representative aspartate surfactants conform to the formula:

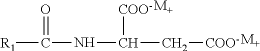

wherein R₁ is a saturated or unsaturated, straight or branched alkyl chain containing 7 to 17 carbon atoms in one aspect and 9 to 13 carbon atoms in another aspect, and M independently is H, sodium, potassium, ammonium or triethanolammonium.

Non-limiting examples of aspartate surfactants are sodium lauroyl aspartate, sodium myristoyl aspartate, sodium cocoyl aspartate, sodium caproyl aspartate, di-sodium lauroyl aspartate, di-sodium myristoyl aspartate, di-sodium cocoyl aspartate, di-sodium caproyl aspartate, potassium lauroyl aspartate, potassium myristoyl aspartate, potassium cocoyl aspartate, potassium caproyl aspartate, di-potassium lauroyl aspartate, di-potassium myristoyl aspartate, di-potassium cocoyl aspartate, di-potassium caproyl aspartate, and mixtures thereof.

In one aspect of the present technology, suitable amphoteric surfactants include but are not limited to alkyl betaines, e.g., lauryl betaine; alkylamido betaines, e.g., cocamidopropyl betaine, lauramidopropyl betaine and cocohexadecyl dimethylbetaine; alkylamido sultaines, e.g., cocamidopropyl hydroxysultaine; (mono- and di-) amphocarboxylates, e.g., sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate; and mixtures thereof.

The foregoing amphoteric surfactants (i.e., the betaines and sultaines are disclosed without a counter ion, as one of ordinary skill in the art will recognize that the under the pH conditions of the compositions containing the amphoteric surfactants, these surfactants are either electrically neutral by virtue of having balanced positive and negative charges, or they contain counter ions such as alkali metal, alkaline earth or ammonium ions as a charge balancing moiety.

In one aspect of the present technology, suitable cationic surfactants include but are not limited to alkylamines, amidoamines, alkyl imidazolines, ethoxylated amines, quaternary compounds, and quaternized esters. In addition, alkylamine oxides can function as a cationic surfactant at a lower pH values.

Non-limiting examples of alkylamines and salts thereof include dimethyl cocamine, dimethyl palmitamine, dioctylamine, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, arachidylbehenylamine, dimethyl lauramine, stearylamine hydrochloride, soyamine chloride, stearylamine formate, N-tallowpropane diamine dichloride, and amodimethicone (INCI name for a silicone polymer and blocked with amino functional groups, such as aminoethylamino propylsiloxane).

Non-limiting examples of amidoamines and salts thereof include stearamido propyl dimethyl amine, stearamidopropyl dimethylamine citrate, palm itam idopropyl diethylamine, and cocamidopropyl dimethylamine lactate.

Non-limiting examples of alkyl imidazoline surfactants include alkyl hydroxyethyl imidazoline, such as stearyl hydroxyethyl imidazoline, coco hydroxyethyl imidazoline, ethyl hydroxymethyl oleyl oxazoline, and the like.

Non-limiting examples of ethyoxylated amines include PEG-cocopolyamine, PEG-15 tallow amine, quaternium-52, and the like.

Exemplary quaternary ammonium surfactants include, but are not limited to cetyl trimethylammonium chloride, cetylpyridinium chloride, dicetyl dimethyl ammonium chloride, dihexadecyl dimethyl ammonium chloride, stearyl dimethyl benzyl ammonium chloride, dioctadecyl dimethyl ammonium chloride, dieicosyl dimethyl ammonium chloride, didocosyl dimethyl ammonium chloride, dihexadecyl dimethyl ammonium chloride, dihexadecyl dimethyl ammonium acetate, behenyl trimethyl ammonium chloride, benzalkonium chloride, benzethonium chloride, and di(cocoalkyl) dimethyl ammonium chloride, ditallowdimethyl ammonium chloride, di(hydrogenated tallow) dimethyl ammonium chloride, di(hydrogenated tallow) dimethyl ammonium acetate, ditallowdimethyl ammonium methyl sulfate, ditallow dipropyl ammonium phosphate, and ditallow dimethyl ammonium nitrate.

At low pH values, alkylamine oxides can protonate and behave similarly to N-alkyl amines. Examples include, but are not limited to, dimethyl-dodecylamine oxide, oleyldi(2-hydroxyethyl) amine oxide, dimethyltetradecylamine oxide, di(2-hydroxyethyl)-tetradecylam ine oxide, dimethylhexadecylamine oxide, behenamine oxide, cocamine oxide, decyltetradecylamine oxide, dihydroxyethyl C₁₂₋₁₅ alkoxypropylamine oxide, dihydroxyethyl cocamine oxide, dihydroxyethyl lauramine oxide, dihydroxyethyl stearamine oxide, dihydroxyethyl tallowamine oxide, hydrogenated palm kernel amine oxide, hydrogenated tallowamine oxide, hydroxyethyl hydroxypropyl C₁₂-C₁₅ alkoxypropylamine oxide, lauramine oxide, myristamine oxide, cetylamine oxide, oleamidopropylamine oxide, oleamine oxide, palmitamine oxide, PEG-3 lauramine oxide, dimethyl lauramine oxide, potassium trisphosphonomethylam ine oxide, soyam idopropylamine oxide, cocamidopropylamine oxide, stearamine oxide, tallowamine oxide, and mixtures thereof.

The nonionic surfactant can be any of the nonionic surfactants known or previously used in the art of aqueous surfactant compositions. Suitable nonionic surfactants include but are not limited to aliphatic C₆ to C₁₈ primary or secondary linear or branched chain acids, alcohols or phenols, linear alcohol and alkyl phenol alkoxylates (especially ethoxylates and mixed ethoxy/propoxy), block alkylene oxide condensate of alkyl phenols, alkylene oxide condensates of alkanols, ethylene oxide/propylene oxide block copolymers, semi-polar nonionics (e.g., amine oxides and phosphine oxides), as well as alkyl amine oxides. Other suitable nonionics include mono or di alkyl alkanolamides and alkyl polysaccharides, sorbitan fatty acid esters, polyoxyethylene sorbitan fatty acid esters, polyoxyethylene sorbitol esters, and polyoxyethylene acids. Examples of suitable nonionic surfactants include coco mono- or diethanolamide, cocamidopropyl and lauramine oxide, polysorbate 20, 40, 60 and 80, ethoxylated linear alcohols, cetearyl alcohol, lanolin alcohol, stearic acid, glyceryl stearate, PEG-150 distearate, PEG-100 stearate, PEG-80 sorbitan laurate, and oleth 20. Other suitable nonionic surfactants include the alkyl glucosides and the alkyl polyglucosides, such as, for example, coco-glucoside, decyl glucoside, lauryl glucoside, decyl diglucoside, lauryl diglucoside and coco diglucoside.

In one aspect, the nonionic surfactant is an alcohol alkoxylate derived from a saturated or unsaturated fatty alcohol containing 8 to 18 carbon atoms, and the number of alkylene oxide groups present in the alcohol range from about 3 to about 12. The alkylene oxide moiety is selected from ethylene oxide, propylene oxide and combinations thereof. In another aspect, the alcohol alkoxylate is derived from a fatty alcohol containing 8 to 15 carbon atoms and contains from 5 to 10 alkoxy groups (e.g. ethylene oxide, propylene oxide, and combinations thereof). Exemplary nonionic fatty alcohol alkoxylate surfactants in which the alcohol residue contains 12 to 15 carbon atoms and contain about 7 ethylene oxide groups are available under the Tomadol® (e.g., product designation 25-7) and Neodol® (e.g., product designation 25-7) trade names from Tomah Products, Inc. and Shell Chemicals, respectively.

An exemplary nonionic alcohol alkoxylated surfactant derived from an unsaturated fatty alcohol and containing about 10 ethylene oxide groups is available from Lubrizol Advanced Materials, Inc. under the trade Chemonic™ oleth-10 ethoxylated alcohol.

Another commercially available alcohol alkoxylate surfactant is sold under the Plurafac® trade name from BASF. The Plurafac surfactants are reaction products of a higher linear alcohol and a mixture of ethylene and propylene oxides, containing a mixed chain of ethylene oxide and propylene oxide, terminated by a hydroxyl group. Examples include C₁₃ to C₁₅ fatty alcohols condensed with 6 moles ethylene oxide and 3 moles propylene oxide, C₁₃ to C₁₅ fatty alcohols condensed with 7 moles propylene oxide and 4 moles ethylene oxide, and C₁₃ to C₁₅ fatty alcohols condensed with 5 moles propylene oxide and 10 moles ethylene oxide.

Another commercially suitable nonionic surfactant is available from Shell Chemicals under the Dobanol™ trade name (product designations 91-5 and 25-7). Product designation 91-5 is an ethoxylated C₉ to C₁₁ fatty alcohol with an average of 5 moles ethylene oxide and product designation 25-7 is an ethoxylated C₁₂ to C₁₅ fatty alcohol with an average of 7 moles ethylene oxide per mole of fatty alcohol.

Other surfactants which can be utilized in the cleansing compositions of the present technology are set forth in more detail in WO 99/21530, U.S. Pat. Nos. 3,929,678, 4,565,647, 5,456,849, 5,720,964, 5,858,948, and 7,115,550, which are herein incorporated by reference. Additionally, suitable surfactants are described in McCutcheon's Emulsifiers and Detergents (North American and International Editions, by Schwartz, Perry and Berch) which is hereby fully incorporated by reference.

The amount of auxiliary surfactant utilized in the cleansing composition is based on the amount of soap present. In one aspect the amount of auxiliary surfactant utilized in the cleansing composition is ranges from about 0, or about 1 to about 30 wt. % (on an active basis) of the weight of the cleansing composition. In another aspect, weight ratio of auxiliary surfactant to soap (calculated on an active weight basis) ranges from about 0:1 to about 2:1, or from about 0.1:1 to about 0.3:1, or from about 0.05:1 to 1.5:1, or 0:0.6, or t 0.05:0.55, or 0.1:0.5.

### Aqueous Phase

The aqueous phase is primarily water, usually deionized or distilled water. In one aspect, the compositions comprise from about 15 to about 90 wt. %, or from about 20 to about 85 wt. %, or from about 35 to about 80 wt. %, or about 40 to about 75 wt. %, or from about 60 to about 70 wt. %, or from about 75 to about 93 wt. %, or from about 80 to about 90 wt. % water, based on the total weight of the composition.

### Optional Components

The personal care cleansing compositions of the present technology can include one or more optional components which are customarily used in the formulation of personal care cleansing products for use on the skin, hair and scalp. Non-limiting examples of such optional components are disclosed in the International Cosmetic Ingredient Dictionary, Fifth Edition, 1993, and the Cosmetic, Toiletry, and Fragrance Association (CTFA) Cosmetic Ingredient Handbook, Second edition, 1992, each of which are incorporated by reference. Exemplary optional components are disclosed below.

### Cationic Polymers

Cationic polymers are components that can enhance the delivery and deposition of conditioning agents and/or provide auxiliary conditioning benefits to the hair, scalp or skin to improve and enhance the conditioning benefits delivered by the compositions of the present technology. Cationic polymer refers to polymers containing at least one cationic moiety or at least one moiety that can be ionized to form a cationic moiety. Typically, these cationic moieties are nitrogen containing groups such as quaternary ammonium or protonated amino groups. The cationic protonated amines can be primary, secondary, or tertiary amines. The cationic polymer typically has a cationic charge density ranging from about 0.2 to about 7 meq/g at the pH of the intended use of the composition. The average molecular weight of the cationic polymer ranges from about 5,000 daltons to about 10,000,000 daltons. Non-limiting examples of such polymers are described in the CTFA International Cosmetic Ingredient Dictionary/Handbook via the CTFA website as well as the CTFA Cosmetic Ingredient Handbook, Ninth Ed., Cosmetic and Fragrance Assn., Inc., Washington D.C. (2002), incorporated herein by reference, can be used.

Suitable cationic polymers can be synthetically derived or natural polymers can be synthetically modified to contain cationic moieties. In one aspect, the cationic polymer contains at least one repeating unit containing a quaternary ammonium salt moiety. Such polymers can be prepared by the polymerization of a diallylamine such as dialkyldiallylammonium salt or copolymer thereof in which the alkyl group contains 1 to about 22 carbon atoms in one aspect and methyl or ethyl in another aspect. Copolymers containing a quaternary moiety derived from a dialkyldiallylammonium salt and an anionic component derived from anionic monomers of acrylic acid and methacrylic acid are suitable conditioning agents. Also suitable are, polyampholyte terpolymers having a cationic component prepared from a derivative of diallylamine, such as a dimethyldiallylammonium salt, an anionic component derived from anionic monomers of acrylic acid or 2-acrylamido-2-methylpropane sulfonic acid and a nonionic component derived from nonionic monomers of acrylamide. The preparation of such quaternary ammonium salt moiety containing polymers can be found, for example, in U.S. Pat. Nos. 3,288,770; 3,412,019; 4,772,462 and 5,275,809, the pertinent disclosures of which are incorporated herein by reference.

In one aspect, suitable cationic polymers include the chloride salts of the foregoing quaternized homopolymers and copolymers in which the alkyl group is methyl or ethyl, and are commercially available under the Merquat® series of trademarks from Lubrizol Advanced Materials, Inc. A homopolymer prepared from diallyl dimethyl ammonium chloride (DADMAC) having the CTFA name, Polyquaternium-6, is available under the Merquat 100 and Merquat 106 trademark. A copolymer prepared from DADMAC and acrylamide having the CTFA name, Polyquaternium-7, is sold under the Merquat 550 trademark. Another copolymer prepared from DADMAC and acrylic acid having the CTFA name, Polyquaternium-22, is sold under the Merquat 280 trademark. The preparation of Polyquaternium-22 and its related polymers is described in U.S. Pat. No. 4,772,462, the pertinent disclosures of which are incorporated herein by reference.

Also useful is an ampholytic terpolymer prepared from a nonionic component derived from acrylamide or methyl acrylate, a cationic component derived from DADMAC or methacrylamidopropyl trimethyl ammonium chloride (MAPTAC), and an anionic component derived from acrylic acid or 2-acrylamido-2-methylpropane sulfonic acid or combinations of acrylic acid and 2-acrylamido-2-methylpropane sulfonic acid. An ampholytic terpolymer prepared from acrylic acid, DADMAC and acrylamide having the CTFA name, Polyquarternium-39, is available under the Merquat Plus 3330 and Mequat 3330PR trademarks. Another ampholytic terpolymer prepared from acrylic acid, methacrylamidopropyl trimethyl ammonium chloride (MAPTAC) and methyl acrylate having the CTFA name, Polyquarternium-47, is available under the Merquat 2001 trademark. Still another ampholytic terpolymer prepared from acrylic acid, MAPTAC and acrylamide having the CTFA name, Polyquarternium-53, is available under the Merquat 2003PR trademark. The preparation of such terpolymers is described in U.S. Pat. No. 5,275,809, the pertinent disclosures of which are incorporated herein by reference.

Exemplary cationically modified natural polymers suitable for use in the hair conditioning composition includes polysaccharide polymers, such as cationically modified cellulose and cationically modified starch derivatives modified with a quaternary ammonium halide moiety. Exemplary cationically modified cellulose polymers are salts of hydroxyethyl cellulose reacted with trimethyl ammonium substituted epoxide (CTFA, Polyquaternium-10). Other suitable types of cationically modified cellulose include the polymeric quaternary ammonium salts of hydroxyethyl cellulose reacted with lauryl dimethyl ammonium substituted epoxide (CTFA, Polyquaternium-24). Cationically modified potato starch having the CTFA name, Starch Hydroxypropyltrimonium Chloride, is available under the Sensomer™ CI-50 trademark, from Lubrizol Advanced Materials, Inc.

Other suitable cationically modified natural polymers include cationic polygalactomannan derivatives such as guar gum derivatives and cassia gum derivatives, e.g., CTFA: Guar Hydroxypropyltrimonium Chloride and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc.

Exemplary cationic polymers and copolymers suitable as conditioners and/or deposition aids in the disclosed technology have the CTFA names Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquarternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-52, Polyquaternium-53, Polyquaternium-54, Polyquarternium-55, Polyquaternium-56, Polyquaternium-57, Polyquaternium-58, Polyquaternium-59, Polyquaternium-60, Polyquaternium-61, Polyquaternium-62, Polyquaternium-63, Polyquaternium-64, Polyquaternium-65, Polyquaternium-66, Polyquaternium-67, Polyquaternium-68, Polyquaternium-69, Polyquaternium-70, Polyquaternium-71, Polyquaternium-72, Polyquaternium-73, Polyquaternium-74, Polyquaternium-75, Polyquaternium-76, Polyquaternium-77, Polyquaternium-78, Polyquaternium-79, Polyquaternium-80, Polyquaternium-81, Polyquaternium-82, Polyquaternium-83, Polyquaternium-84, Polyquaternium-85, Polyquaternium-86, Polyquaternium-87, and mixtures thereof.

The cationic compounds can be present from about 0.05 to about 5 wt. % percent, or from about 0.1 to about 3 wt. %, or from about 0.5 to about 2.0 wt. % (based on the total weight of the composition).

### Auxiliary Rheology Modifiers

The compositions of the present technology can be thickened by using a thickener in the external aqueous phase. The non-polar oil phase of the emulsion may be thickened with waxes, hydrophobically modified metal oxides, and layered silicates and aluminates such as fumed silica, fumed alumina, and smectite clays. The compositions of the present technology may further comprise a suspending agent at concentrations effective for suspending water insoluble material in dispersed form in the compositions or for modifying the viscosity of the composition. Thickeners and suspending agents useful in the present technology in the aqueous phase include anionic polymers and nonionic polymers. Exemplary rheology modifiers include acrylic based polymers and copolymers. One class of acrylic based rheology modifiers are the carboxyl functional alkali-swellable and alkali-soluble thickeners (ASTs) produced by the free-radical polymerization of acrylic acid alone or in combination with other ethylenically unsaturated monomers. The polymers can be synthesized by solvent/precipitation as well as emulsion polymerization techniques. Exemplary synthetic rheology modifiers of this class include homopolymers of acrylic acid or methacrylic acid and copolymers polymerized from one or more monomers of acrylic acid, substituted acrylic acid and C₁-C₃₀ alkyl esters of acrylic acid. Substituted acrylic acid contains a substituent positioned on the alpha and/or beta carbon atom of the molecule wherein the substituent is preferably and independently selected from C₁₋₄ alkyl, —CN, and —COON. Optionally, other ethylenically unsaturated monomers such as, for example, styrene, vinyl acetate, ethylene, butadiene, acrylonitrile, as well as mixtures thereof can be copolymerized into the backbone. The foregoing polymers are optionally crosslinked by a monomer that contains two or more moieties that contain ethylenic unsaturation. In one aspect, the crosslinker is selected from a polyalkenyl polyether of a polyhydric alcohol containing at least two alkenyl ether groups per molecule. Other Exemplary crosslinkers are selected from allyl ethers of sucrose and allyl ethers of pentaerythritol, and mixtures thereof. These polymers are more fully described in U.S. Pat. Nos. 5,087,445; 4,509,949; and 2,798,053 herein incorporated by reference.

In one aspect, the AST rheology modifier or thickener is a crosslinked homopolymer polymerized from acrylic acid or methacrylic acid and is generally referred to under the INCI name of Carbomer. Commercially available Carbomers include Carbopol® polymers 934, 940, 941, 956, 980 and 996 available from Lubrizol Advanced Materials, Inc. In another aspect, the AST rheology modifier is selected from a crosslinked emulsion copolymer polymerized from a first monomer selected from one or more monomers of (meth)acrylic acid, substituted acrylic acid, and salts of (meth)acrylic acid and substituted acrylic acid and a second monomer selected from one or more C₁-C₅ alkyl acrylate esters of (meth)acrylic acid. These polymers are designated under the INCI name of Acrylates Copolymer. Acrylates Copolymers are commercially available under the trade names Aculyn® 33 from Rohm and Haas and Carbopol® Aqua SF-1 from Lubrizol Advanced Materials, Inc. In another aspect, the rheology modifier is selected from a crosslinked copolymer polymerized from a first monomer selected from one or more monomers of acrylic acid, substituted acrylic acid, salts of acrylic acid and salts of substituted acrylic acid and a second monomer selected from one or more C₁₀-C₃₀ alkyl acrylate esters of acrylic acid or methacrylic acid. In one aspect, the monomers can be polymerized in the presence of a steric stabilizer such as disclosed in U.S. Pat. No. 5,288,814, which is herein incorporated by reference. Some of the forgoing polymers are designated under INCI nomenclature as Acrylates/C10-30 Alkyl Acrylate Crosspolymer and are commercially available under the trade names Carbopol® 1342 and 1382, Carbopol® Ultrez 20 and 21, Carbopol® ETD 2020 and Pemulen® TR-1 and TR-2 from Lubrizol Advanced Materials, Inc.

Another class of rheology modifiers and thickeners suitable for use in the present technology includes amphiphilically modified ASTs commonly referred to as hydrophobically modified alkali-swellable and/or alkali-soluble emulsion (HASE) polymers. Typical HASE polymers are free radical addition emulsion polymers polymerized from pH sensitive or anionic monomers (e.g., acrylic acid and/or methacrylic acid), hydrophobic monomers (e.g., C₁-C₃₀ alkyl esters of acrylic acid and/or methacrylic acid, acrylonitrile, styrene), an “amphiphilic monomer”, and an optional crosslinking monomer. The amphiphilic monomer comprises an ethylenically unsaturated polymerizable end group, a non-ionic hydrophilic midsection that is terminated by a hydrophobic end group. The non-ionic hydrophilice midsection comprises a polyoxyalkylene group, e.g., polyethylene oxide, polypropylene oxide, or mixtures of polyethylene oxide/polypropylene oxide segments. The terminal hydrophobic end group is typically a C₈-C₄₀ aliphatic moiety. Exemplary aliphatic moieties are selected from linear and branched alkyl substituents, linear and branched alkenyl substituents, carbocyclic substituents, aryl substituents, aralkyl substituents, arylalkyl substituents, and alkylaryl substituents. In one aspect, amphiphilic monomers can be prepared by the condensation (e.g., esterification or etherification) of a polyethoxylated and/or polypropoxylated aliphatic alcohol (typically containing a branched or unbranched C₈-C₄₀ aliphatic moiety) with an ethylenically unsaturated monomer containing a carboxylic acid group (e.g., acrylic acid, methacrylic acid), an unsaturated cyclic anhydride monomer (e.g., maleic anhydride, itaconic anhydride, citraconic anhydride), a monoethylenically unsaturated monoisocyanate (e.g., a,a-dimethyl-m-isopropenyl benzyl isocyanate) or an ethylenically unsaturated monomer containing a hydroxyl group (e.g., vinyl alcohol, allyl alcohol). Polyethoxylated and/or polypropoxylated aliphatic alcohols are ethylene oxide and/or propylene oxide adducts of a monoalcohol containing the C₈-C₄₀ aliphatic moiety. Non-limiting examples of alcohols containing a C₈-C₄₀ aliphatic moiety are capryl alcohol, iso-octyl alcohol (2-ethyl hexanol), pelargonic alcohol (1-nonanol), decyl alcohol, lauryl alcohol, myristyl alcohol, cetyl alcohol, cetyl alcohol, cetearyl alcohol (mixture of C₁₆-C₁₈ monoalcohols), stearyl alcohol, isostearyl alcohol, elaidyl alcohol, oleyl alcohol, arachidyl alcohol, behenyl alcohol, lignoceryl alcohol, ceryl alcohol, montanyl alcohol, melissyl, lacceryl alcohol, geddyl alcohol, and C₂-C₂₀ alkyl substituted phenols (e.g., nonyl phenol), and the like.

Exemplary HASE polymers are disclosed in U.S. Pat. Nos. 3,657,175; 4,384,096; 4,464,524; 4,801,671; and 5,292,843, which are herein incorporated by reference. In addition, an extensive review of HASE polymers is found in Gregory D. Shay, Chapter 25, “Alkali-Swellable and Alkali-Soluble Thickener Technology A Review”, Polymers in Aqueous Media—Performance Through Association, Advances in Chemistry Series 223, J. Edward Glass (ed.), ACS, pp. 457-494, Division Polymeric Materials, Washington, D.C. (1989), the relevant disclosures of which are incorporated herein by reference. The HASE polymers are commercially available from Lubrizol Advanced Materials, Inc. under the trade designation Novethix198 L-10 polymer (INCI Name: Acrylates/Beheneth-25 Methacrylate Copolymer and Rohm & Haas under the trade designations Aculyn™ 22 (INCI Name: Acrylates/Steareth-20 Methacrylate Copolymer), Aculyn™ 44 (INCI Name: PEG-150/Decyl Alcohol/SMDI Copolymer), Aculyn 46™ (INCI Name: PEG-150/Stearyl Alcohol/SMDI Copolymer), and Aculyn™ 88 (INCI Name: Acrylates/Steareth-20 Methacrylate Crosspolymer).

Hydrophobically modified alkoxylated methyl glucoside, such as, for example, PEG-120 Methyl Glucose Dioleate, PEG-120 Methyl Glucose Trioleate, and PEG-20 Methyl Glucose Sesquistearate, available from Lubrizol Advanced Materials, Inc., under the trade names, Glucamate™ DOE-120, Glucamate™ LT, and Glucamate™ SSE-20, respectively, are also suitable rheology modifiers.

Polysaccharides obtained from tree and shrub exudates, such as gum Arabic, gum gahatti, and gum tragacanth, as well as pectin; seaweed extracts, such as alginates and carrageenans; algae extracts, such as agar; microbial polysaccharides, such as xanthan, gellan, and wellan; cellulose ethers, such as ethylhexylethylcellulose, hydroxybutylmethylcellulose, hydroxyethylmethylcellulose, hydroxypropylmethylcellulose, methylcellulose, carboxymethylcellulose, hydroxyethylcellulose, and hydroxypropylcellulose; polygalactomannans, such as fenugreek gum, cassia gum, locust bean gum, tara gum, and guar gum; starches, such as corn starch, tapioca starch, rice starch, wheat starch, potato starch and sorghum starch can also be employed in the present technology as suitable thickeners and rheology modifiers.

### Humectants

Suitable humectants include allantoin; pyrrolidonecarboxylic acid and its salts; hyaluronic acid and its salts; sorbic acid and its salts, salicylic acid and its salts; urea, hydroxyethyl urea; lysine, arginine, cystine, guanidine, and other amino acids; polyhydroxy alcohols such as glycerin, propylene glycol, hexylene glycol, hexanetriol, ethoxydiglycol, dimethicone copolyol, and sorbitol, and the esters thereof; polyethylene glycol; glycolic acid and glycolate salts (e.g. ammonium and quaternary alkyl ammonium); lactic acid and lactate salts (e.g. ammonium and quaternary alkyl ammonium); sugars and starches; sugar and starch derivatives (e.g. alkoxylated methyl glucose ethers, such as PPG-20 methyl glucose ether); D-panthenol; lactamide monoethanolamine; acetamide monoethanolamine; and the like, and mixtures thereof. Preferred humectants include the C₃ to C₆ diols and triols, such as glycerin, propylene glycol, 1,3-propanediol, hexylene glycol, hexanetriol, and the like, and mixtures thereof. Such suitable humectants typically comprise from about 1 wt. % to about 10 wt. % in one aspect, from about 2 wt. % to about 8 wt. % in another aspect, and from about 3 wt. % to about 5 wt. % in a further aspect of the present technology, based on the total weight of the surfactant containing composition.

### Fragrances and Perfumes

Exemplary perfumes, fragrances and fragrance oils include but are not limited to allyl cyclohexane propionate, ambrettolide, Ambrox® DL (dodecahydro-3a,6,6,9a-tetramethylnaphtho[2,1-b]furan), amyl benzoate, amyl cinnamate, amyl cinnamic aldehyde, amyl salicylate, anethol, aurantiol, benzophenone, benzyl butyrate, benzyl iso-valerate, benzyl salicylate, cadinene, campylcyclohexal, cedrol, cedryl acetate, cinnamyl cinnamate, citronellyl acetate, citronellyl isobutyrate, citronellyl propionate, cuminic aldehyde, cyclohexylsalicylate, cyclamen aldehyde, cyclomyral, dihydro isojasmonate, diphenyl methane, diphenyl oxide, dodecanal, dodecalactone, ethylene brassylate, ethylmethyl phenylglycidate, ethyl undecylenate, exaltolide, Galoxilide® (1,3,4,6,7,8-hexhydro,4,6,6,7,8,8-hexamethyl-cyclopenta-γ-2-benzopyran), geranyl acetate, geranyl isobutyrate, hexadecanolide, hexenyl salicylate, hexyl cinnamic aldehyde, hexyl salicylate, α-ionone, β-ionone, γ-ionone, α-irone, isobutyl benzoate, isobutyl quinoline, Iso E Super® (7-acetyl,1,2,3,4,5,6,7,8-octahydro,1,1,6,7-tetramethyl napthalene), cis-jasmone, lilial, linalyl benzoate, 20 methoxy naphthaline, methyl cinnamate, methyl eugenol, γ-methylionone, methyl linolate, methyl linolenate, musk indanone, musk ketone, musk tibetine, myristicin, neryl acetate, δ-nonalactone, γ-nonalactone, patchouli alcohol, phantolide, phenylethyl benzoate, phenylethylphenylacetate, 2-phenylethanol, phenyl heptanol, phenyl hexanol, α-santalol, thibetolide, tonalid, δ-undecalactone, γ-undecalactone, vertenex, vetiveryl acetate, yara-yara, ylangene, allo-ocimene, allyl caproate, allyl heptoate, anisole, camphene, carvacrol, carvone, citral, citronellal, citronellol, citronellyl nitrile, coumarin, cyclohexyl ethylacetate, p-cymene, decanal, dihydromyrcenol, dihydromyrcenyl acetate, dimethyl octanol, ethyllinalool, ethylhexyl ketone, eucalyptol, fenchyl acetate, geraniol, gernyl formate, hexenyl isobutyrate, hexyl acetate, hexyl neopentanoate, heptanal, isobornyl acetate, isoeugenol, isomenthone, isononyl acetate, isononyl alcohol, isomenthol, isopulegol, limonene, linalool, linalyl acetate, menthyl acetate, methyl chavicol, methyl octyl acetaldehyde, myrcene, napthalene, nerol, neral, nonanal, 2-nonanone, nonyl acetate, octanol, octanal, α-pinene, β-pinene, rose oxide, α-terpinene, γ-terpinene, α-terpinenol, terpinolene, terpinyl acetate, tetrahydrolinalool, tetrahydromyrcenol, undecenal, veratrol, verdox, acetanisol; amyl acetate; anisic aldehyde; anisylalcohol; benzaldehyde; benzyl acetate; benzyl acetone; benzyl alcohol; benzyl formate; hexenol; laevo-carveol; d-carvone; cinnamaldehyde; cinnamic alcohol; cinnamyl acetate; cinnamyl formate; cis-3-hexenyl acetate; Cyclal C (2,4-dimethyl-3-cyclohexen-1-carbaldehyde); dihydroxyindole; dimethyl benzyl carbinol; ethyl acetate; ethyl acetoacetate; ethyl butanoate; ethyl butyrate; ethyl vanillin; tricyclo decenyl propionate; furfural; hexanal; hexenol; hydratropic alcohol; hydroxycitronellal; indole; isoamyl alcohol; isopulegyl acetate; isoquinoline; ligustral; linalool oxide; methyl acetophenone; methyl amyl ketone; methyl anthranilate; methyl benzoate; methyl benzyl acetate; methyl heptenone; methyl heptyl ketone; methyl phenyl carbinyl acetate; methyl salicylate; octalactone; para-cresol; para-methoxy acetophenone; para-methyl acetophenone; phenethylalcohol; phenoxy ethanol; phenyl acetaldehyde; phenyl ethyl acetate; phenyl ethyl alcohol; prenyl acetate; propyl butyrate; safrole; vanillin and viridine.

Amounts of each of the fragrance or perfume components may range from about 0.000001 to about 2 wt. %, or from 0.00001 to about 1.5 wt. %, or from 0.0001 to about 1 wt. %, or from about 0.001 to about 0.8 wt. %, based on of the weight of the composition.

### Botanicals

The compositions of the present technology can include water soluble or oil soluble botanical materials extracted from a particular plant, fruit, nut, or seed. Suitable botanicals can include, for example, Aloe barbadensis leaf juice, Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, sage, strawberry, sweet pea, tomato, vanilla fruit, comfrey, arnica, centella asiatica, cornflower, horse chestnut, ivy, magnolia, oat, pansy, skullcap, seabuckthorn, white nettle, and witch hazel. Botanicals include, for example, chlorogenic acid, glutathione, glycrrhizin, neohesperidin, quercetin, rutin, morin, myricetin, absinthe, and chamomile.

Botanicals can be present in an amount ranging from about 0.001 to about 10 wt. %, or from about 0.005 to about 8 wt. %, or from about 0.01 to about 5 wt. %, based of the total weight of the composition.

### Vitamins

The composition of the present technology can include a vitamin(s). Illustrative vitamins are vitamin A (retinol), vitamin B₂, vitamin B₃ (niacinamide), vitamin B₆, vitamin C, vitamin E, folic acid and biotin. Derivatives of the vitamins may also be employed. For instance, vitamin C derivatives include ascorbyl tetraisopalmitate, magnesium ascorbyl phosphate and ascorbyl glycoside. Derivatives of vitamin E include tocopheryl acetate, tocopheryl palm itate and tocopheryl linoleate. DL-panthenol and derivatives may also be employed.

The total amount of vitamins when present in compositions according to the present technology may range from about 0.001 to about 10 wt. %, or from 0.01 to about 1 wt. %, or from 0.1 to about 0.5 wt. %, based on the weight of the total composition.

### Chelating Agents

The composition of the present technology can include a chelating agent(s). Suitable chelators include EDTA (ethylene diamine tetraacetic acid) and salts thereof such as disodium EDTA and tetrasodium ETDA, citric acid and salts thereof, tetrasodium glutamate diacetate, cyclodextrins, and the like, and mixtures thereof.

Chelating agents typically comprise from about 0.001 to about 3 wt. %, or from about 0.01 to about 2 wt. %, or from about 0.01 to about 1 wt. %, based on the total weight of the surfactant containing composition.

### Preservatives

The composition of the present technology can include a preservative(s). Preservatives include compounds that have antifungal activity, antimicrobial activity, antioxidant activity, UV protection activity, and the like. Non-limiting examples of suitable preservatives include polymethoxy bicyclic oxazolidine, methylparaben, propylparaben, ethylparaben, butylparaben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzophenone-4, dibutylhydroxytoluene (BHT), benzoisothiazolinone, triclosan, quaternium-15, salicylic acid salts, and the like, and mixtures thereof.

The preservative(s) is typically presenent from about 0.01 to about 3.0 wt. %, or from about 0.1 to about 1 wt. %, or from about 0.3 to about 1 wt. %, based on the total weight of the composition.

### pH Adjusting Agents

In one aspect, the pH of the compositions of the present technology range from about 7 and above, or from about 7 to about 14, or from about 7.2, 7.3, 7.4 7.5, 7.6, 7,7, or 7.8 to about 12, or from about 8 to about 11, or from about 8.5 to about 10.

An alkaline material can be incorporated in the compositions of the disclosed technology to raise the pH of the composition to desired levels. Any material capable of increasing the pH of the composition is suitable, including inorganic and organic bases, and combinations thereof. Examples of inorganic bases include but are not limited to the alkali metal hydroxides (especially sodium, potassium, and ammonium), and alkali metal carbonates such as sodium carbonate. Examples of organic bases include but are not limited to triethanolamine (TEA), diisopropanolamine, triisopropanolamine, am inomethyl propanol, dodecylamine, cocamine, oleamine, morpholine, triamylamine, triethylamine, tetrakis(hydroxypropyl)ethylenediamine, L-arginine, tromethamine (2-amino 2-hydroxymethyl-1,3-propanediol), and PEG-15 cocamine.

Acidic materials can be incorporated into the compositions of the present technology to decrease the pH of the composition to a desired pH level. Such acidic materials include organic acids and inorganic acids, for example, acetic acid, citric acid, fumaric acid, tartaric acid, alpha-hydroxy acids, beta-hydroxy acids, amino acids, salicylic acid, lactic acid, glycolic acid, and natural fruit acids, or inorganic acids, for example, hydrochloric acid, nitric acid, sulfuric acid, sulfamic acid, phosphoric acid, and combinations thereof.

Buffering agents can be used in the compositions of the disclosed technology. Suitable buffering agents include, but are not limited to, alkali or alkali earth metal carbonates, phosphates, bicarbonates, citrates, borates, acetates, acid anhydrides, succinates, including sodium phosphate, sodium citrate, sodium acetate, sodium bicarbonate, and sodium carbonate.

The following examples further describe and demonstrate embodiments within the scope of the present technology. These examples are presented solely for the purpose of illustration and are not to be construed as limitations of the present technology since many variations thereof are possible without departing from the spirit and scope thereof. Unless otherwise specified weight percent (wt. %) is given in weight percent, based on the weight of the total composition.

### Ingredient Descriptions and Abbreviations

| | |
|---|---|
| AM (E-Sperse ® RS-1618) | Amphiphilic crosslinker with two polymerizable reactive groups from |
| | Ethox Chemical, LLC |
| AOS | Sodium C₁₄₋₁₆ Alpha Olefin Sulfonate (40% Active) |
| APE | Allyl Pentaerythritol |
| BEM | Sipomer ® Polyethoxylated (25 moles) Behenyl Methacrylate (66.67% |
| | BEM/33.33% MAA by wt.), Rhodia |
| Carbopol ® SF-1 Polymer | INCI Name: Acrylates Copolymer, Lubrizol Advanced Materials, Inc. |
| Emulsion | (30% active solids) |
| DI Water | Deionized Water |
| Ethoxylated MEG Ester | Glucamate ™ VLT Liquid Thickener, INCI Name: PEG-120 Methyl |
| (EMegE) | Glucose Trioleate (and) Propanediol (68-72% active), Lubrizol |
| | Advanced Materials, Inc. |
| EA | Ethyl Acrylate |
| HEMA | 2-Hydroxyethyl Methacrylate |
| n-BA | n-Butyl Acrylate |
| Merquat ™ 3330PR | INCI Name: Polyquaternium-39, Lubrizol Advanced Materials, Inc. |
| Polymer | |
| Neolone ™ 950 | INCI Name: Methylisothiazolinone, Dow Chemical Company |
| Preservative | |
| Selvol ® 203 PVA | Polyvinyl Alcohol (hydrolysis % = 87-89%), Sekisui Corporation |
| Selvol ® 502 PVA | Polyvinyl Alcohol (hydrolysis % = 87-89%), Sekisui Corporation |
| SLS | Sulfochem ™ Sodium Lauryl Sulfate (anionic surfactant), Lubrizol |
| | Advanced Materials, Inc. (30% active) |
| TBHP | t-butyl hydroperoxide (70%), Alfa Aesar |
| VA-086 | Azo VA-086 2,2′-Azobis[2-methyl-N-(2-hydroxyethyl)propionamide], |
| | Wako |

### EXAMPLE 1

Monomer Composition=EA/n-BA/HEMA/BEM (35/15/45/5 wt. % Total Monomers) and Crosslinked with 0.08wt % APE (Based on the Weight of the Dry Polymer)

A monomer pre-mix was made by mixing 140 grams of DI water, 3.75 grams of 40% alpha olefin sulfonate (AOS) aqueous solution, 175 grams of EA, 71 grams of n-BA, 33.33 grams of BEM and 225 grams of HEMA. Initiator A was made by mixing 2.86 grams of 70% TBHP in 40 grams of DI water. Reductant A was prepared by dissolving 0.13 grams of erythorbic acid in 5 grams of DI water. Reductant B was prepared by dissolving 2.0 grams of erythorbic acid in 100 grams of DI water. A 3-liter reactor vessel was charged with 800 grams of DI water, 10 grams of 40% AOS and 25 grams of Selvol® 502 PVA and then was heated to 65° C. under a nitrogen blanket and proper agitation. Initiator A was then added to the reaction vessel and followed by adding reductant A. After about 1 minute, the monomer pre-mix was metered into the reaction vessel over a period of 150 minutes; simultaneously, reductant B was metered into the reaction vessel over a period of 180 minutes. After the addition of monomer pre-mix, a solution of 0.40 grams of 70% APE and 3.6 grams n-BA was added into the monomer pre-mixer. After the completion of monomer pre-mix feed, 33 grams of DI water was added to flush the residual monomers from the pre-mixer. After the completion of reductant B feed, the temperature of the reaction vessel was maintained at 65° C. for 65 minutes. The reaction vessel was then cooled to 60° C. A solution of 1.79 grams of 70% TBHP and 0.13 grams of 40% AOS in 25 grams of DI water was added to the reaction vessel. After 5 minutes, a solution of 1.05 grams of erythorbic acid in 25 grams of DI water was added to the reaction vessel. After 30 minutes, a solution of 1.79 grams of 70% TBHP and 0.13 grams of 40% AOS in 25 grams of DI water was added to the reaction vessel. After 5 minutes, a solution of 1.05 grams of erythorbic acid in 25 grams of DI water was added to the reaction vessel. The reaction vessel was maintained at 60° C. for about 30 minutes. Then, the contents of the reaction vessel were cooled to room temperature and filtered through 100 μm cloth. The pH of the resulting emulsion was adjusted to 4 with 28% ammonium hydroxide.

### EXAMPLE 2

### Monomer Composition=EA/n-BA/HEMA/BEM (20.5/27.5/45/5) (wt. % Total Monomers)

An emulsion polymer was prepared as follows. A monomer pre-mix was made by mixing 140 grams of DI water, 5 grams of E-Sperse 1618 (anionic reactive surfactant), 102.5 grams of (EA), 137.5 grams of (n-BA), 46.67 grams of (BEM), and 225 grams of (HEMA). Initiator A was made by mixing 5 grams of VA-086 in 40 grams of DI water. Initiator B was made by mixing 2.5 grams of VA-086 in 100 grams of DI water. A 3-liter reactor vessel was charged with 770 grams of DI water, 10 grams of Selvol® 203 PVA and 6 grams of SLS, and then was heated to 85° C. under a nitrogen blanket and proper agitation. Initiator A was initially added to the reaction vessel. After about 1 minute, the monomer pre-mix was metered into the reaction vessel over a period of 120 minutes; simultaneously, initiator B was metered into the reaction vessel over a period of 150 minutes. After the completion of monomer pre-mix feed, 33 grams of DI water was added to flush the residual monomers in the pre-mixer. After the completion of initiator B feed, the temperature of the reaction vessel was maintained at 85° C. for 60 minutes. The reaction vessel was then cooled to 49° C. A solution of 0.6 grams of 70% TBHP and 16.8 grams of DI water was added to the reaction vessel. After 30 seconds, a solution of 0.59 grams of erythorbic acid in 16.8 grams of DI water was added to the reaction vessel. After 30 minutes, a solution of 0.6 grams of 70% TBHP and 16.8 grams of DI water was added to the reaction vessel. After 30 seconds, a solution of 0.59 grams of erythorbic acid in 16.8 grams of DI water was added to the reaction vessel. The reaction vessel was maintained at 49° C. for about 60 minutes and 340 grams of DI water was added into the reactor. Then, the reaction vessel was cooled to room temperature and the polymer emulsion was filtered through 100 micron cloth. The resulting polymer emulsion had a solids content of 25.4%, and a polymer particle size of 82 nm.

### EXAMPLE 3

Cleansing compositions were prepared using different non-polar oil phases utilizing the components set forth in Table 1.

TABLE 1

| | Formulation 1 | Formulation 2 | Formulation 3 |
|---|---|---|---|
| Component | (wt. %) | (wt. %) | (wt. %) |
| DI Water | 35.00 | 25.00 | 15.00 |
| Glycerin | 3.00 | 3.00 | 3.00 |
| Polymer of Example 2 | 10.00 | 10.00 | 10.00 |
| (25% active solids) | | | |
| Potassium Cocoate | 30.00 | 30.00 | 30.00 |
| (39% active) | | | |
| Lauramidopropyl | 10.00 | 10.00 | 10.00 |
| betaine (30% TS) | | | |
| Mineral Oil | 20.00 | — | — |
| Petrolatum, | — | 20.00 | — |
| White USP | | | |
| Silicone Oil (PDMS | — | — | 20.00 |
| 4,000 Daltons) | | | |
| Phenoxyethanol | 0.50 | 0.50 | 0.50 |
| Merquat | 2.00 | 2.00 | 2.00 |
| 3300PR (10%) | | | |
| Final pH | 9.52 | 9.66 | 9.51 |
| Brookfield Viscosity¹ | 10,980 | 9,840 | 4,420 |
| (mPa · s) | | | |

¹Brookfield DVII+ viscometer @ 25° C. (spindle sized as appropriate)

500 g of each formulation was prepared by adding the components shown in the order listed in the table with mixing using an overhead mixer equipped with a marine blade at 300 rpm. The petrolatum was heated to 50° C. prior to addition. After each addition of a component, the formulation was mixed until homogeneous. Subsequently, the formulations were removed from the mixer and placed in glass sample vessels to monitor sample stability at 45° C. If visible separation of the oil and water phases was observed in the final formulation at any time during 3 months of stability testing, the sample was deemed to have failed the stability test. All formulations were found to be stable, passing visual stability evaluations after 3 months at 45° C. The stability of Formulations 1, 2, and 3 demonstrates the ability of the formulations to remain phase stable across a range of non-polar oils.

### EXAMPLE 4

Cleansing compositions are prepared utilizing the components set forth the in Table 2.

TABLE 2

| | Formulation 4 | Formulation 5 | Formulation 6 |
|---|---|---|---|
| Component | (wt. %) | (wt. %) | (wt. %) |
| DI Water | 35.00 | 25.00 | 15.00 |
| Glycerin | 3.00 | 3.00 | 3.00 |
| Polymer of Example 1 | 10.00 | 10.00 | 10.00 |
| (25% active solids) | | | |
| Potassium Cocoate | 30.00 | 30.00 | 30.00 |
| (39% active) | | | |
| Lauramidopropyl | 10.00 | 10.00 | 10.00 |
| betaine (30% TS) | | | |
| Mineral Oil | 20.00 | — | — |
| Petrolatum, | — | 20.00 | |
| White USP | | | |
| Silicone Oil (PDMS | — | | 20.00 |
| 4,000 Daltons) | | | |
| Phenoxyethanol | 0.50 | 0.50 | 0.50 |
| Merquat | 2.00 | 2.00 | 2.00 |
| 3300PR (10%) | | | |

500 g of each formulation is prepared by adding the components shown in the order listed in the table with mixing using an overhead mixer equipped with a marine blade at 300 rpm. (The petrolatum may be pre-headed to 50° C. to ease addition). After each addition of a component, the formulation is mixed until homogeneous. The pH of each formulation can be adjusted by adding citric acid to attain the desired pH. The formulation is then removed from the mixer and is placed in glass sample vessels to evaluate sample stability at 45° C. as set forth in Example 3.

### EXAMPLE 5

Liquid soap compositions are identically prepared utilizing the ingredients in Table 3.

TABLE 3

| | | Formulation | Formulation | Formulation |
|---|---|---|---|---|
| | | 7 | 9 | 11 |
| Part | Component | (wt. %) | (wt. %) | (wt. %) |
| A | Lauric acid | 5 | 5 | 5 |
| | Myristic acid | 5 | 5 | 5 |
| | Palmitic acid | 5 | 5 | 5 |
| B | Polymer of | 10.00 | 10.00 | 10.00 |
| | Example 1 | | | |
| | (25% active solids) | | | |
| | DI Water | 10 | 10 | 10 |
| C | DI Water | 27.60 | 27.60 | 27.60 |
| | Disodium EDTA | 0.1 | 0.1 | 0.1 |
| | Glycerin | 5 | 5 | 5 |
| | Potassium | 4.15 | 4.15 | 4.15 |
| | Hydroxide | | | |
| | (85% active) | | | |
| D | Merquat 3330PR | 2 | 2 | 2 |
| | (10% active) | | | |
| | Sodium | 3 | 3 | 3 |
| | Lauroamphoacetate | | | |
| | (37% active) | | | |
| | Cocamidopropyl | 3 | 3 | 3 |
| | Betaine | | | |
| | (35% TS) | | | |
| | Fragrance | 0.1 | 0.1 | 0.1 |
| | Neolone ™ 950 | 0.05 | 0.05 | 0.05 |
| | Presevative | | | |
| | Mineral Oil | 20.00 | — | — |
| | Petrolatum, White | — | 20.00 | — |
| | USP | | | |
| | Silicone Oil, 4,000 | — | — | 20.00 |
| | MW | | | |

Part A is prepared by heating the fatty acids at 80° C. in a water bath until melted and uniform. Part B is separately prepared by combining a latex polymer with DI water. The ingredients in part C are added one at a time into a separate beaker in the order listed while mixing with an overhead mixer equipped with a marine blade at 150 rpm until uniform. Part C is then added to part A while mixing with a marine blade at 150 rpm. After 30 minutes of mixing the formulation is removed from the heat and allowed to cool to 60° C. with mixing. Once a temperature of 60° C. is reached, Part B is added to the combined parts A and C and the mixing speed is increased to 300 rpm. The formula is then mixed for 30 minutes and then the mixing speed would be decreased to 150 rpm. Once the temperature decreases to 50° C., Merquat™ 3330PR polymer is added, and the formulation is allowed to cool further while mixing. Once the temperature reaches 40° C., the remaining components in Part D are added one by one in the order listed with mixing after each addition until a uniform mixture is obtained. (The petrolatum may be pre-headed to 50° C. to ease addition). Once the oil is added, the mixing speed is increased to 300 rpm, and the composition is allowed to mix for 20 minutes. The formulation is then removed from the mixer and is placed in glass sample vessels to evaluate sample stability at 45° C. as set forth in Example 3.

1. A detersive composition comprising:
a. water
b. from about 1 to about 5 wt. %, or from about 1.5 to about 3 wt. %, or from about 2 to about 2.5 wt. % (based on the weight of the composition) of a crosslinked amphiphilic nonionic polymer prepared from a monomer mixture comprising:
i. 35% to about 55%, or from about 40% to about 50% or from about 42% to about 48%, or from about 44% to about 46% by weight of at least one C₁ to C₅ hydroxyalkyl ester of (meth)acrylic acid (based on the total weight of the monounsaturated monomers);
ii. from about 10% to about 50%, or from about 10% to about 40%, or from about 12% to about 35%, or from about 15 to about 25% by weight of at least one C₁ to C₅ alkyl ester of (meth)acrylic acid;
iii. from about 0.1% to about 20%, or from about 0.5% to about 18%, or from about 1%, 2%, 3%, 4%, 5%, 6%, 7%, 8%, 9% or 10% to about 15% by weight of an associative monomer (wherein all monomer weight percentages under I, ii, and iii are based on the total weight of the monounsaturated monomers); and
iv. from about 0.1, 0.2, 0.3, 0.4, 0.5, 0.6, 0.7, 0.8, 0.9 or 1 to about 5 parts by wt. of at least one polyunsaturated crosslinker monomer (based on 100 parts by wt. of the monounsaturated monomers);
c. from about10 to about 45 wt. %, or from about 12 to about 40 wt. %, or from about 15 to about 35 wt. %, or from about 18 to about 30 wt. %, or from about 20 to about 25 wt. %, of a non-polar oil phase based on the total weight of the composition;
d. from about 5 to about 40 wt. %, or from about 8 to about 30 wt. %, or from about 10 to about 25 wt. %, of at least one fatty acid soap, based on the total weight of the composition;
e. optionally at least one surfactant other than d).

2. A detersive composition of claim 1, wherein said non-polar oil phase is selected from a hydrocarbon oil, petrolatum, silicone oil, and mixtures thereof.

3. A detersive composition of claim 1, wherein said non-polar hydrocarbon oil is selected from a non-polar volatile hydrocarbon oil, a non-polar non-volatile hydrocarbon oil, and mixtures thereof.

4. A detersive composition of claim 1, wherein said non-polar volatile hydrocarbon oil is selected from linear or branched C₅-C₂₀ alkanes, and mixtures thereof.

5. A detersive composition of claim 1, wherein said non-polar, non-volatile hydrocarbon oil is selected from linear or branched paraffinic hydrocarbons and olefins containing at least 20 carbon atoms.

6. A detersive composition of claim 1, wherein said non-polar hydrocarbon oil is selected from C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, petrolatum, pentahydrosqualene, squalene, squalane, and mixtures thereof.

7. A detersive composition of claim 1, wherein said non-polar oil is a non-volatile silicone oil selected from polyalkylsiloxanes, polyarylsiloxanes, polyalkylarylsiloxanes, and mixtures thereof.

8. A detersive composition of claim 1, wherein said non-polar oil is selected from mineral oil, petrolatum, polydimethylsiloxane, and mixtures thereof.

9. A detersive composition of claim 1, wherein said at least one fatty acid soap is selected from the alkali metal and/or ethanolamine salt of a C₈ to C₂₂ fatty acid.

10. A detersive composition of claim 1, wherein said at least one fatty acid soap is selected from a salt of octanoic acid, decanoic acid, lauric acid, myristic acid, pentadecanoic acid, palmitic acid, margaric acid, steric acid, isostearic acid, nonadecanoic acid, arachidic acid, behenic acid, and mixtures thereof.

11. A detersive composition of claim 1, wherein said at least one fatty acid soap is selected from a mixture of fatty acid soaps comprising lauric acid salts, myristic acid salts and palmitic acid salts.

12. A detersive composition of claim 1, wherein said at least one fatty acid soap is a cocoate salt.

13. A detersive composition of claim 1, wherein said fatty acid soap is an alkali metal or alkanol ammonium salt of said fatty acid.

14. A detersive composition of claim 1, wherein said composition further comprises a component selected from a humectant, a hair conditioner, a skin conditioner, a fragrance agent, a preservative, a colorant, a botanical extract, a chelating agent, a pH adjusting agent, a thickening agent, and combinations thereof.

15. A detersive composition of claim 1, wherein said crosslinked amphiphilic nonionic polymer is prepared from a monomer mixture comprising:
i. from about 42 to about 49 wt. % of 2-hydroxyethyl methacrylate;
ii. from about 15 to about 40 wt. % of ethyl acrylate;
iii. from about 12 to about 30 wt. % of butyl acrylate;
iv. from about 5 to about 15 wt. % of behenyl ethoxylated methacrylate; and
v. from about 0.1 to about 1 part by wt. of at least one polyunsaturated crosslinker monomer (based on 100 parts by wt. of the monounsaturated monomers utilized to prepare the polymer).

16. A detersive composition of claim 1, wherein said crosslinked amphiphilic nonionic polymer is prepared from a monomer mixture comprising:
i. about 45% by weight of 2-hydroxyethyl methacrylate;
ii. about 20.5% by weight of ethyl acrylate;
iii. about 27.5% by weight of butyl acrylate;
iv. about 7% by weight of behenyl ethoxylated methacrylate (based on 100 parts by wt. of the monounsaturated monomers utilized to prepare the polymer); and
v. from about 0.1 to about 1 part by wt. of at least one polyunsaturated crosslinker monomer (based on 100 parts by wt. of the monounsaturated monomers utilized to prepare the polymer.

17. A detersive composition of claim 1, wherein said crosslinked amphiphilic nonionic polymer is prepared from a monomer mixture comprising:
i. about 45% by weight of 2-hydroxyethyl methacrylate;
ii. about 15% by weight of ethyl acrylate;
iii. about 25% by weight of butyl acrylate;
iv. about 15% by weight of behenyl ethoxylated methacrylate (based on 100 parts by wt. of the monounsaturated monomers utilized to prepare the polymer); and
v. from about 0.5 to about 2 parts by wt. of at least one polyunsaturated amphiphilic crosslinker monomer (based on 100 parts by wt. of the monounsaturated monomers utilized to prepare the polymer).

18. A detersive composition of claim 1, wherein said at least one polyunsaturated amphiphilic crosslinker monomer is represented by the structure: 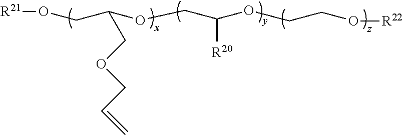
where R²¹ is a C₁₀₋₂₄ alkyl, alkaryl, alkenyl, or cycloalkyl, R²⁰═CH₃, CH₂CH₃, C₆H₅, or C₁₄H₂₉; x is 2-10, y is 0-200, z is 4-200, from about 5 to 60 in another aspect, and from about 5 to 40 in a further aspect; and R²² is H or Z⁻ M⁺ Z can be either SO₃⁻ or PO₃²⁻, and M⁺ is Na⁺, K⁺, NH₄⁺, or an alkanolamine such as, for example, monoethanolamine, diethanolamine, and triethanolamine.

19. A detersive composition of claim 1, wherein said at least one polyunsaturated amphiphilic crosslinker monomer is represented by the structure: 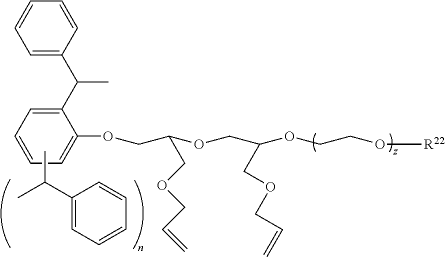
where n is 1 or 2; z is 4 to 40, or 5 to 38, or 10 to 20; and R²² is H, SO₃⁻M⁺ or PO₃⁻² M⁺, and M is selected from Na, K, and NH₄.

20. A detersive composition of claim 1, wherein said at least one polyunsaturated amphiphilic crosslinker monomer is represented by the structure:

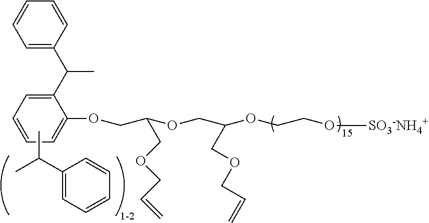

21. A detersive composition of claim 1, wherein said composition has a pH from about 7 to about 14, or from about 7.2, 7.3, 7.4 7.5, 7.6, 7,7, or 7.8 to about 12, or from about 8 to about 11, or from about 8.5 to about 10.

22. A detersive composition of claim 1 further comprising a cationic conditioning polymer selected from Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquarternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-42, Polyquaternium-43, Polyquaternium-44, Polyquaternium-45, Polyquaternium-46, Polyquaternium-47, Polyquaternium-48, Polyquaternium-49, Polyquaternium-50, Polyquaternium-51, Polyquaternium-52, Polyquaternium-53, Polyquaternium-54, Polyquarternium-55, Polyquaternium-56, Polyquaternium-57, Polyquaternium-58, Polyquaternium-59, Polyquaternium-60, Polyquaternium-61, Polyquaternium-62, Polyquaternium-63, Polyquaternium-64, Polyquaternium-65, Polyquaternium-66, Polyquaternium-67, Polyquaternium-68, Polyquaternium-69, Polyquaternium-70, Polyquaternium-71, Polyquaternium-72, Polyquaternium-73, Polyquaternium-74, Polyquaternium-75, Polyquaternium-76, Polyquaternium-77, Polyquaternium-78, Polyquaternium-79, Polyquaternium-80, Polyquaternium-81, Polyquaternium-82, Polyquaternium-83, Polyquaternium-84, Polyquaternium-85, Polyquaternium-86, Polyquaternium-87, and mixtures thereof.

23. A detersive composition of claim 1 further comprising an auxiliary synthetic surfactant other than a fatty acid soap selected from an anionic surfactant, an amphoteric surfactant, and mixtures thereof.

24. A detersive composition of claim 1, wherein the weight ratio of auxiliary surfactant to fatty acid soap (calculated on an active weight basis) ranges from about 0:1 to about 2:1, or from about 0.1:1 to about 0.3:1, or from about 0.05:1 to 1.5:1, or 0:0.6, ort 0.05:0.55, or 0.1:0.5.

25. A detersive composition of claim 1 which exhibits stability upon storage for at least 4 weeks at 5° C., 4 weeks at 25° C. and 4 weeks at 50° C.

26. A method for simultaneously cleansing and conditioning a keratinous substrate comprising the steps of:
i) applying a composition of claim 1 to a keratinous substrate;
ii) applying a shear force to the composition applied to said keratinous substrate sufficient to generate a foam; and
iii) rinsing said foamed composition from said keratinous substrate.
